(19) United States
(12) Patent Application Publication
(10) Pub. No.: US 2021/0052218 A1
(43) Pub. Date: Feb. 25, 2021

(54) SYSTEMS AND METHODS FOR SEPSIS DETECTION AND MONITORING

(71) Applicant: Patchd, Inc., San Francisco, CA (US)

(72) Inventors: Robert QUINN, San Francisco, CA (US); Wei-Jien TAN, San Francisco, CA (US)

(21) Appl. No.: 16/993,076

(22) Filed: Aug. 13, 2020

Related U.S. Application Data
(60) Provisional application No. 62/889,456, filed on Aug. 20, 2019.

(51) Int. Cl. A61B 5/00; A61B 5/0408; A61B 5/024; A61B 5/145; A61B 5/01; G06N 3/08

(52) U.S. Cl. CPC A61B 5/4842; A61B 5/0006; A61B 5/0408; A61B 5/746; G06N 3/08; A61B 5/02405; A61B 5/14542; A61B 5/14532; A61B 5/01; A61B 5/742

(57) ABSTRACT

The present disclosure provides systems and methods for collecting and analyzing vital sign information to predict a likelihood of a subject having a disease or disorder. In an aspect, a system for monitoring a subject may comprise: sensors comprising an electrocardiogram (ECG) sensor, which sensors are configured to acquire health data comprising vital sign measurements of the subject over a period of time; and a mobile electronic device, comprising: an electronic display; a wireless transceiver; and one or more computer processors configured to (i) receive the health data from the sensors through the wireless transceiver, (ii) process the health data using a trained algorithm to generate an output indicative of a progression or regression of a health condition of the subject over the period of time at a sensitivity of at least about 80%, and (iii) provide the output for display to the subject on the electronic display.

### CROSS-REFERENCE

This application claims the benefit of U.S. Provisional Patent Application No. 62/889,456, filed Aug. 20, 2019, which is incorporated by reference herein in its entirety.

### BACKGROUND

Patient monitoring may require collection and analysis of vital sign information over a period of time to detect clinical signs of the patient having occurrence or recurrence of a disease or disorder. However, patient monitoring outside of a clinical setting (e.g., a hospital) may pose challenges for non-invasive collection of vital sign information and accurate prediction of occurrence or recurrence of an adverse health condition such as deterioration or occurrence or recurrence of a disease or disorder.

### SUMMARY

Sepsis is one of the leading causes of mortality in U.S. hospitals, with an estimated 1.7 million annual cases, of which 270 thousand end in death. Sepsis may generally refer to “the dysregulated host response to infection.” Previously, sepsis had been defined as the presence of both infection and the systemic inflammatory response with septic shock being the presence of sepsis and organ dysfunction. Further, hospital costs associated with admissions of sepsis patients can increase with increasing severity of the condition, costing about $16 thousand, about $25 thousand, and about $38 thousand for cases of sepsis without organ dysfunction, severe sepsis, and septic shock, respectively. While the problem of sepsis in an inpatient and critical care setting is monumental, the beginnings of sepsis are often present before admission. For example, about 80% of sepsis cases are present at hospital admission. Therefore, there exists a need for sepsis detection in an outpatient setting. In addition, sepsis is a particularly important problem in certain disease states. The relative risk for a cancer patient in contracting sepsis is nearly 4 times that of non-cancer patients and as high as 65 times in patients with myeloid leukemia patients. While the impacts of sepsis are most apparent in the highly increased risk of mortality in an acute setting, sepsis can also significantly impact long-term outcomes.

Recognized herein is the need for systems and methods for patient monitoring by continuous collection and analysis of vital sign information. Such analysis of vital sign information (e.g., heart rate and/or blood pressure) of a subject (patient) may be performed by a wearable monitoring device (e.g., at the subject's home, instead of a clinical setting such as a hospital) over a period of time to predict a likelihood of the subject having an adverse health condition (e.g., deterioration of the patient's state, occurrence or recurrence of a disease or disorder (e.g., sepsis), or occurrence of a complication.

The present disclosure provides systems and methods that may advantageously collect and analyze vital sign information over a period of time to accurately and non-invasively predict a likelihood of the subject having an adverse health condition (e.g., deterioration of the patient's state, occurrence or recurrence of a disease or disorder (e.g., sepsis), or occurrence of a complication). Such systems and methods may allow patients with elevated risk of an adverse health condition such as deterioration or a disease or disorder to be accurately monitored for deterioration, occurrence, or recurrence outside of a clinical setting. In some embodiments, the systems and methods may process health data including collected vital sign information or other clinical health data (e.g., obtained by blood testing, imaging, etc.).

In an aspect, the present disclosure provides a system for monitoring a subject, comprising: one or more sensors comprising an electrocardiogram (ECG) sensor, which one or more sensors are configured to acquire health data comprising a plurality of vital sign measurements of the subject over a period of time; and a mobile electronic device, comprising: an electronic display; a wireless transceiver; and one or more computer processors operatively coupled to the electronic display and the wireless transceiver, which one or more computer processors are configured to (i) receive the health data from the one or more sensors through the wireless transceiver, (ii) process the health data using a trained algorithm to generate an output indicative of a progression or regression of a health condition of the subject over the period of time at a sensitivity of at least about 80%, and (iii) provide the output for display to the subject on the electronic display.

In some embodiments, the ECG sensor comprises one or more ECG electrodes. In some embodiments, the ECG sensor comprises two or more ECG electrodes. In some embodiments, the ECG sensor comprises no more than three ECG electrodes.

In some embodiments, the plurality of vital sign measurements comprises one or more vital sign measurements selected from the group consisting of heart rate, heart rate variability, blood pressure (e.g., systolic and diastolic), respiratory rate, blood oxygen concentration (SpO₂), carbon dioxide concentration in respiratory gases, a hormone level, sweat analysis, blood glucose, body temperature, impedance (e.g., bioimpedance), conductivity, capacitance, resistivity, electromyography, galvanic skin response, neurological signals (e.g., electroencephalography), immunology markers, and other physiological measurements. In some embodiments, the plurality of vital sign measurements comprises heart rate or heart rate variability. In some embodiments, the plurality of vital sign measurements comprises blood pressure (e.g., systolic and diastolic).

In some embodiments, the wireless transceiver comprises a Bluetooth transceiver. In some embodiments, the wireless transceiver comprises a cellular radio transceiver (e.g., 3G, 4G, LTE, or 5G). In some embodiments, the one or more computer processors are further configured to store the acquired health data in a database. In some embodiments, the health condition is sepsis. In some embodiments, the one or more computer processors are further configured to present an alert on the electronic display based at least on the output. In some embodiments, the one or more computer processors are further configured to transmit an alert over a network to a health care provider of the subject based at least on the output. In some embodiments, the trained algorithm comprises a machine learning based classifier configured to process the health data to generate the output indicative of the progression or regression of the health condition of the subject. In some embodiments, the machine learning-based classifier is selected from the group consisting of a support vector machine (SVM), a naïve Bayes classification, a random forest, a neural network, a deep neural network (DNN), a recurrent neural network (RNN), a deep RNN, a long short-term memory (LSTM) recurrent neural network (RNN), and a gated recurrent unit (GRU) recurrent neural network (RNN). In some embodiments, the trained algorithm comprises a recurrent neural network (RNN) or a long short-term memory (LSTM) recurrent neural network (RNN). In some embodiments, the trained algorithm comprises a long short-term memory (LSTM) recurrent neural network (RNN). In some embodiments, the subject has undergone an operation. In some embodiments, the operation is surgery, and the subject is being monitored for post-surgery complications. In some embodiments, the subject has received a treatment comprising a bone marrow transplant or active chemotherapy. In some embodiments, the subject is being monitored for post-treatment complications.

In some embodiments, the one or more computer processors are configured to process the health data using the trained algorithm to generate the output indicative of the progression or regression of the health condition of the subject over the period of time with a sensitivity of at least about 75%, wherein the period of time includes a window beginning about 2 hours, about 4 hours, about 6 hours, about 8 hours, or about 10 hours prior to the onset of the health condition and ending at the onset of the health condition. In some embodiments, the period of time includes a window beginning about 4 hours prior to the onset of the health condition and ending at about 2 hours prior to the onset of the health condition. In some embodiments, the period of time includes a window beginning about 6 hours prior to the onset of the health condition and ending at about 4 hours prior to the onset of the health condition. In some embodiments, the period of time includes a window beginning about 8 hours prior to the onset of the health condition and ending at about 6 hours prior to the onset of the health condition. In some embodiments, the period of time includes a window of about 1 hour, about 2 hours, about 3 hours, about 4 hours, about 5 hours, about 6 hours, about 7 hours, about 8 hours, about 10 hours, about 12 hours, about 14 hours, about 16 hours, about 18 hours, about 20 hours, about 22 hours, or about 24 hours prior to the onset of the health condition. For example, for a window of about 5 hours, the period of time can be from about 5 hours prior to the onset of the health condition to the onset of the health condition, from about 7 hours prior to the onset of the health condition to about 2 hours prior to the onset of the health condition, from about 9 hours prior to the onset of the health condition to about 4 hours prior to the onset of the health condition, from about 11 hours prior to the onset of the health condition to about 6 hours prior to the onset of the health condition, etc. In some embodiments, the one or more computer processors are configured to process the health data using the trained algorithm to generate the output indicative of the progression or regression of the health condition of the subject over the period of time with a sensitivity of at least about 75%, wherein the period of time includes a window beginning about 10 hours prior to the onset of the health condition and ending at about 8 hours prior to the onset of the health condition. In some embodiments, the one or more computer processors are configured to process the health data using the trained algorithm to generate the output indicative of the progression or regression of the health condition of the subject over the period of time with a specificity of at least about 40%. In some embodiments, the specificity is at least about 50%.

In some embodiments, the plurality of vital sign measurements comprises no more than 10 types of vital sign measurements. In some embodiments, the plurality of vital sign measurements comprises no more than 6 types of vital sign measurements. In some embodiments, the plurality of vital sign measurements comprises no more than 10 types of vital sign measurements selected from the group consisting of heart rate, heart rate variability, systolic blood pressure, diastolic blood pressure, respiratory rate, blood oxygen concentration (SpO₂), carbon dioxide concentration in respiratory gases, a hormone level, sweat analysis, blood glucose, body temperature, impedance, conductivity, capacitance, resistivity, electromyography, galvanic skin response, neurological signals, and immunology markers. In some embodiments, the plurality of vital sign measurements comprises no more than 6 types of vital sign measurements selected from the group consisting of heart rate, heart rate variability, systolic blood pressure, diastolic blood pressure, respiratory rate, blood oxygen concentration (SpO₂), carbon dioxide concentration in respiratory gases, a hormone level, sweat analysis, blood glucose, body temperature, impedance, conductivity, capacitance, resistivity, electromyography, galvanic skin response, neurological signals, and immunology markers. In some embodiments, the plurality of vital sign measurements comprises no more than 6 types of vital sign measurements, wherein the 6 types of vital sign measurements are heart rate, respiratory rate, body temperature, systolic blood pressure, diastolic blood pressure, and blood oxygen.

In some embodiments, (b) comprises using the trained algorithm to generate the output indicative of the progression or regression of the health condition of the subject over the period of time at an Area Under the Receiver Operating Characteristic (AUROC) of at least 0.70. In some embodiments, (b) comprises using the trained algorithm to generate the output indicative of the progression or regression of the health condition of the subject over the period of time at an Area Under the Receiver Operating Characteristic (AUROC) of at least about 0.85. In some embodiments, (b) comprises using the trained algorithm to generate the output indicative of the progression or regression of the health condition of the subject over the period of time at an Area Under the Receiver Operating Characteristic (AUROC) of at least about 0.70, wherein the period of time includes a window beginning about 8 hours prior to the onset of the health condition and ending at the onset of the health condition. In some embodiments, (b) comprises using the trained algorithm to generate the output indicative of the progression or regression of the health condition of the subject over the period of time at an Area Under the Precision-Recall Curve (AUPRC) of at least 0.40, wherein the period of time includes a window beginning about 8 hours prior to the onset of the health condition and ending at the onset of the health condition. In some embodiments, (b) comprises using the trained algorithm to generate the output indicative of the progression or regression of the health condition of the subject over the period of time at an Area Under the Precision-Recall Curve (AUPRC) of at least about 0.65, wherein the period of time includes a window beginning about 8 hours prior to the onset of the health condition and ending at the onset of the health condition.

In another aspect, the present disclosure provides a method for monitoring a subject, comprising: (a) receiving, using a wireless transceiver of a mobile electronic device of the subject, health data from one or more sensors, which one or more sensors comprise an electrocardiogram (ECG) sensor, which health data comprises a plurality of vital sign measurements of the subject over a period of time; (b) using one or more programmed computer processors of the mobile electronic device to process the health data using a trained algorithm to generate an output indicative of a progression or regression of a health condition of the subject over the period of time at a sensitivity of at least about 80%; and (c) presenting the output for display on an electronic display of the mobile electronic device.

In some embodiments, the ECG sensor comprises one or more ECG electrodes. In some embodiments, the ECG sensor comprises two or more ECG electrodes. In some embodiments, the ECG sensor comprises no more than three ECG electrodes.

In some embodiments, the plurality of vital sign measurements comprises one or more measurements selected from the group consisting of heart rate, heart rate variability, blood pressure (e.g., systolic and diastolic), respiratory rate, blood oxygen concentration (SpO₂), carbon dioxide concentration in respiratory gases, a hormone level, sweat analysis, blood glucose, body temperature, impedance (e.g., bioimpedance), conductivity, capacitance, resistivity, electromyography, galvanic skin response, neurological signals (e.g., electroencephalography), immunology markers, and other physiological measurements. In some embodiments, the plurality of vital sign measurements comprises heart rate or heart rate variability. In some embodiments, the plurality of vital sign measurements comprises blood pressure (e.g., systolic and diastolic).

In some embodiments, the wireless transceiver comprises a Bluetooth transceiver. In some embodiments, the wireless transceiver comprises a cellular radio transceiver (e.g., 3G, 4G, LTE, or 5G). In some embodiments, the processor is further configured to store the acquired health data in a database. In some embodiments, the health condition is sepsis. In some embodiments, the method further comprises presenting an alert on the electronic display based at least on the output. In some embodiments, the method further comprises transmitting an alert over a network to a health care provider of the subject based at least on the output. In some embodiments, processing the health data comprises using a machine learning based classifier to generate the output indicative of the progression or regression of the health condition of the subject. In some embodiments, the machine learning-based classifier is selected from the group consisting of a support vector machine (SVM), a naïve Bayes classification, a random forest, a neural network, a deep neural network (DNN), a recurrent neural network (RNN), a deep RNN, a long short-term memory (LSTM) recurrent neural network (RNN), and a gated recurrent unit (GRU) recurrent neural network (RNN). In some embodiments, the trained algorithm comprises a recurrent neural network (RNN). In some embodiments, the subject has undergone an operation. In some embodiments, the operation is surgery, and the subject is being monitored for post-surgery complications. In some embodiments, the subject has received a treatment comprising a bone marrow transplant or active chemotherapy. In some embodiments, the subject is being monitored for post-treatment complications.

In some embodiments, (b) comprises processing the health data using the trained algorithm to generate the output indicative of the progression or regression of the health condition of the subject over the period of time with a sensitivity of at least about 75%, wherein the period of time includes a window beginning about 2 hours, about 4 hours, about 6 hours, about 8 hours, or about 10 hours prior to the onset of the health condition and ending at the onset of the health condition. In some embodiments, the period of time includes a window beginning about 4 hours prior to the onset of the health condition and ending at about 2 hours prior to the onset of the health condition. In some embodiments, the period of time includes a window beginning about 6 hours prior to the onset of the health condition and ending at about 4 hours prior to the onset of the health condition. In some embodiments, the period of time includes a window beginning about 8 hours prior to the onset of the health condition and ending at about 6 hours prior to the onset of the health condition. In some embodiments, the period of time includes a window of about 1 hour, about 2 hours, about 3 hours, about 4 hours, about 5 hours, about 6 hours, about 7 hours, about 8 hours, about 10 hours, about 12 hours, about 14 hours, about 16 hours, about 18 hours, about 20 hours, about 22 hours, or about 24 hours prior to the onset of the health condition. For example, for a window of about 5 hours, the period of time can be from about 5 hours prior to the onset of the health condition to the onset of the health condition, from about 7 hours prior to the onset of the health condition to about 2 hours prior to the onset of the health condition, from about 9 hours prior to the onset of the health condition to about 4 hours prior to the onset of the health condition, from about 11 hours prior to the onset of the health condition to about 6 hours prior to the onset of the health condition, etc. In some embodiments, (b) comprises processing the health data using the trained algorithm to generate the output indicative of the progression or regression of the health condition of the subject over the period of time with a sensitivity of at least about 75%, wherein the period of time includes a window beginning about 10 hours prior to the onset of the health condition and ending at the onset of the health condition. In some embodiments, (b) comprises processing the health data using the trained algorithm to generate the output indicative of the progression or regression of the health condition of the subject over the period of time with a specificity of at least about 40%. In some embodiments, the specificity is at least about 50%.

In some embodiments, a system is provided for monitoring a subject, comprising: the system; a digital processing device comprising: a processor, an operating system configured to perform executable instructions, a memory, and a computer program including instructions executable by the digital processing device to create an application analyzing the acquired health data to generate an output indicative of a progression or regression of a health condition of the subject over a period of time at a sensitivity of at least about 80%, the application comprising: a software module applying a trained algorithm to the acquired health data to generate the output indicative of the progression or regression of the health condition of the subject over a period of time at a sensitivity of at least about 75%. In some embodiments, the trained algorithm comprises a machine learning based classifier configured to process the health data to generate the output indicative of the progression or regression of the health condition of the subject. In some embodiments, the health condition is sepsis.

In another aspect, the present disclosure provides a system for monitoring a subject, comprising: a communications interface in network communication with a mobile electronic device of a user, wherein the communication interface receives from the mobile electronic device health data collected from a subject using one or more sensors, which one or more sensors comprise an electrocardiogram (ECG) sensor, wherein the health data comprises a plurality of vital sign measurements of the subject over a period of time; one or more computer processors operatively coupled to the communications interface, wherein the one or more computer processors are individually or collectively programmed to (i) receive the health data from the communications interface, (ii) use a trained algorithm to analyze the health data to generate an output indicative of a progression or regression of a health condition of the subject over the period of time at a sensitivity of at least about 75%, and (iii) direct the output to the mobile electronic device over the network. In some embodiments, the trained algorithm comprises a machine learning based classifier configured to process the health data to generate the output indicative of the progression or regression of the health condition of the subject. In some embodiments, the health condition is sepsis.

In another aspect, the present disclosure provides a system for monitoring a subject for an onset or progression of sepsis, comprising one or more sensors configured to acquire health data comprising a plurality of vital sign measurements of the subject over a period of time; a wireless transceiver; and one or more computer processors configured to (i) receive the health data from the one or more sensors through the wireless transceiver, and (ii) process the health data using a trained algorithm to generate an output indicative of the onset or progression of sepsis of the subject at a sensitivity of at least about 75%. In some embodiments, the one or more computer processors are part of an electronic device separate from the one or more sensors. In some embodiments, the electronic device is a mobile electronic device.

In another aspect, the present disclosure provides a method for monitoring a subject for an onset or progression of sepsis, comprising (a) using one or more sensors to acquire health data comprising a plurality of vital sign measurements of the subject over a period of time; (b) using an electronic device in wireless communication with the one or more sensors to receive the health data from the one or more sensors; and (c) processing the health data using a trained algorithm to generate an output indicative of the onset or progression of sepsis of the subject at a sensitivity of at least about 75%. In some embodiments, the one or more sensors are separate from the electronic device. In some embodiments, the electronic device is a mobile electronic device. In some embodiments, the health data is processed by the electronic device. In some embodiments, the health data is processed by a computer system separate from the electronic device. In some embodiments, the computer system is a distributed computer system in network communication with the electronic device.

In another aspect, the present disclosure provides a method for monitoring a subject, comprising: (a) receiving health data comprising a plurality of vital sign measurements of the subject over a period of time; (b) processing the health data with a trained computer algorithm to generate an output indicative of a progression or regression of sepsis of the subject over the period of time at a sensitivity of at least about 80%; and (c) presenting the output for display on an electronic display.

In some embodiments, the plurality of vital sign measurements comprises one or more vital sign measurements selected from the group consisting of heart rate, heart rate variability, systolic blood pressure, diastolic blood pressure, respiratory rate, blood oxygen concentration (SpO₂), carbon dioxide concentration in respiratory gases, a hormone level, sweat analysis, blood glucose, body temperature, impedance, conductivity, capacitance, resistivity, electromyography, galvanic skin response, neurological signals, and immunology markers.

In some embodiments, the method further comprises presenting an alert on the electronic display when the output is indicative of a progression of sepsis of the subject. In some embodiments, the method further comprises transmitting an alert over a network to a health care provider of the subject based at least on the output.

In some embodiments, processing the health data comprises using a machine learning-based classifier to generate the output indicative of the progression or regression of the sepsis of the subject. In some embodiments, the machine learning-based classifier is selected from the group consisting of a support vector machine (SVM), a naïve Bayes classification, a random forest, a neural network, a deep neural network (DNN), a recurrent neural network (RNN), a deep RNN, a long short-term memory (LSTM) recurrent neural network (RNN), and a gated recurrent unit (GRU) recurrent neural network (RNN). In some embodiments, the trained algorithm comprises a recurrent neural network (RNN) or a long short-term memory (LSTM) recurrent neural network (RNN). In some embodiments, the trained algorithm comprises a long short-term memory (LSTM) recurrent neural network (RNN).

In some embodiments, the subject has undergone an operation or has been admitted into an intensive care unit (ICU). In some embodiments, the operation is surgery, and the subject is being monitored for post-surgery complications. In some embodiments, the subject has received a treatment comprising a bone marrow transplant or active chemotherapy. In some embodiments, the subject is being monitored for post-treatment complications.

In some embodiments, (b) comprises processing the health data using the trained algorithm to generate the output indicative of the progression or regression of the sepsis of the subject over the period of time with a sensitivity of at least about 75%, wherein the period of time includes a window beginning about 2 hours prior to the onset of the sepsis and ending at the onset of the sepsis. In some embodiments, the period of time includes a window beginning about 4 hours prior to the onset of the sepsis and ending at about 2 hours prior to the onset of the sepsis. In some embodiments, the period of time includes a window beginning about 6 hours prior to the onset of the sepsis and ending at about 4 hours prior to the onset of the sepsis. In some embodiments, the period of time includes a window beginning about 8 hours prior to the onset of the sepsis and ending at about 6 hours prior to the onset of the sepsis. In some embodiments, (b) comprises processing the health data using the trained algorithm to generate the output indicative of the progression or regression of the sepsis of the subject over the period of time with a sensitivity of at least about 75%, wherein the period of time includes a window beginning about 10 hours prior to the onset of the sepsis and ending at about 8 hours prior to the onset of the sepsis. In some embodiments, (b) comprises processing the health data using the trained algorithm to generate the output indicative of the progression or regression of the health condition of the subject over the period of time with a specificity of at least about 40%. In some embodiments, the specificity is at least about 50%.

In some embodiments, the plurality of vital sign measurements comprises no more than 10 types of vital sign measurements. In some embodiments, the plurality of vital sign measurements comprises no more than 6 types of vital sign measurements. In some embodiments, the plurality of vital sign measurements comprises no more than 10 types of vital sign measurements selected from the group consisting of heart rate, heart rate variability, systolic blood pressure, diastolic blood pressure, respiratory rate, blood oxygen concentration (SpO₂), carbon dioxide concentration in respiratory gases, a hormone level, sweat analysis, blood glucose, body temperature, impedance, conductivity, capacitance, resistivity, electromyography, galvanic skin response, neurological signals, and immunology markers. In some embodiments, the plurality of vital sign measurements comprises no more than 6 types of vital sign measurements selected from the group consisting of heart rate, heart rate variability, systolic blood pressure, diastolic blood pressure, respiratory rate, blood oxygen concentration (SpO₂), carbon dioxide concentration in respiratory gases, a hormone level, sweat analysis, blood glucose, body temperature, impedance, conductivity, capacitance, resistivity, electromyography, galvanic skin response, neurological signals, and immunology markers. In some embodiments, the plurality of vital sign measurements comprises no more than 6 types of vital sign measurements, wherein the 6 types of vital sign measurements are heart rate, respiratory rate, body temperature, systolic blood pressure, diastolic blood pressure, and blood oxygen.

In some embodiments, (b) comprises using the trained algorithm to generate the output indicative of the progression or regression of the sepsis of the subject over the period of time at an Area Under the Receiver Operating Characteristic (AUROC) of at least 0.70. In some embodiments, (b) comprises using the trained algorithm to generate the output indicative of the progression or regression of the sepsis of the subject over the period of time at an Area Under the Receiver Operating Characteristic (AUROC) of at least about 0.85. In some embodiments, (b) comprises using the trained algorithm to generate the output indicative of the progression or regression of the sepsis of the subject over the period of time at an Area Under the Receiver Operating Characteristic (AUROC) of at least about 0.70, wherein the period of time includes a window beginning about 8 hours prior to the onset of the sepsis and ending at the onset of the sepsis. In some embodiments, (b) comprises using the trained algorithm to generate the output indicative of the progression or regression of the sepsis of the subject over the period of time at an Area Under the Precision-Recall Curve (AUPRC) of at least 0.40, wherein the period of time includes a window beginning about 8 hours prior to the onset of the sepsis and ending at the onset of the sepsis. In some embodiments, (b) comprises using the trained algorithm to generate the output indicative of the progression or regression of the sepsis of the subject over the period of time at an Area Under the Precision-Recall Curve (AUPRC) of at least about 0.65, wherein the period of time includes a window beginning about 8 hours prior to the onset of the sepsis and ending at the onset of the sepsis.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 illustrates an overview of the system architecture.

FIG. 2 illustrates an example of the data flows in the system architecture.

FIG. 3 is a technical illustration of the exterior of the device enclosure.

FIG. 4 is a technical illustration of the interior components of the device enclosure.

FIG. 5 illustrates an example of an electronic system diagram of the device.

FIG. 6 illustrates three ECG electrode cables, which may correspond to two inputs into a differential amplifier and a reference right-leg-drive electrode providing noise cancellation.

FIG. 7 illustrates example mockups of the application graphical user interface (GUI).

FIG. 8 shows a computer system that is programmed or otherwise configured to implement methods provided herein.

FIG. 9 illustrates an example of an algorithm architecture comprising a long short-term memory (LSTM) recurrent neural network (RNN).

FIG. 10 illustrates an example of defining sepsis onset, such that suspicion of sepsis infection is considered to be present when antibiotics administration and bacterial cultures are performed within a defined time period. This figure shows that suspicion of infection is defined to occur at the time of the first of two events (e.g., an antibiotics administration that is followed by a bacterial culture performed within 48 hours, or a bacterial culture performed that is followed by an antibiotics administration within 72 hours). Once suspicion of infection is confirmed, sepsis onset is defined when the SOFA score increases by 2 or more relative to the start of a 24-hour window afterward.

FIG. 11 illustrates an age distribution histogram of a selected cohort.

FIG. 12 illustrates a machine learning algorithm for predicting sepsis from normalized vital signs, comprising a temporal extraction engine, a prediction engine, and a prediction layer.

FIG. 13A illustrates an area under the precision-recall (PR) curve vs. time. FIG. 13B illustrates an area under the receiver operator characteristic (ROC) curve vs. time. FIGS. 13C-13D illustrate precision-recall (PR) and receiver operating characteristic (ROC) curves, respectively, plotted at different times for a sepsis prediction algorithm vs. the prediction made by the Sequential Organ Failure Assessment (SOFA) score at the onset of sepsis. Note that the sepsis prediction algorithm generates an ROC that is comparable to the existing measures, the SOFA score and modified early warning score (MEWS).

FIG. 14 illustrates a general model architecture of the deep learning algorithm (DLA). The model comprises four components. The first component comprises the input component, where vital signs and demographic information are normalized and fed into the models as an input vector. The second component comprises a recurrent neural network (RNN) layer to model the time-dependent relationships within the data, in which stacked long short-term memory (LSTM) layers are used. The third component comprises a set of dense layers, where the representations of the data from the recurrent neural networks are combined together. The number of hidden units and layers may be tuned as hyper-parameters. The fourth component comprises a prediction layer, which determines a prediction indicative of whether a patient is sepsis-positive or sepsis-negative.

FIGS. 15A-15B illustrate a comparison in performance between the deep learning algorithm (DLA) and a set of four risk score approaches to predicting sepsis onset (MEWS, SOFA, qSOFA (quick SOFA), and SIRS (Systemic Inflammatory Response Syndrome)). FIG. 15A illustrates plots of Area Under the Receiver Operating Characteristic (AUROC) vs. time (left) and Area Under the Precision-Recall Curve (AUPRC) vs. time (right) for the DLA and four risk score approaches (MEWS, SIRS, SOFA, and qSOFA). FIG. 15B illustrates a receiver-operator characteristic (ROC) curve (left) and a precision-recall curve (PRC) (right) for the DLA plotted at sepsis onset and at 8 hours before, as well as the comparison to the four risk score approaches to predicting sepsis onset (MEWS, SOFA, qSOFA, and SIRS) onset.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Various terms used throughout the present description may be read and understood as follows, unless the context indicates otherwise: “or” as used throughout is inclusive, as though written “and/or”; singular articles and pronouns as used throughout include their plural forms, and vice versa; similarly, gendered pronouns include their counterpart pronouns so that pronouns should not be understood as limiting anything described herein to use, implementation, performance, etc. by a single gender; “exemplary” should be understood as “illustrative” or “exemplifying” and not necessarily as “preferred” over other embodiments. Further definitions for terms may be set out herein; these may apply to prior and subsequent instances of those terms, as will be understood from a reading of the present description. Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “subject,” as used herein, generally refers to a human such as a patient. The subject may be a person (e.g., a patient) with a disease or disorder, or a person that has been treated for a disease or disorder, or a person that is being monitored for recurrence of a disease or disorder, or a person that is suspected of having the disease or disorder, or a person that does not have or is not suspected of having the disease or disorder. The disease or disorder may be an infectious disease, an immune disorder or disease, a cancer, a genetic disease, a degenerative disease, a lifestyle disease, an injury, a rare disease, or an age related disease. The infectious disease may be caused by bacteria, viruses, fungi and/or parasites. For example, the disease or disorder may comprise sepsis, atrial fibrillation, stroke, heart attack, and other preventable outpatient illnesses. For example, the disease or disorder may comprise deterioration or recurrence of a disease or disorder for which the subject has previously been treated.

Patient monitoring may require collection and analysis of vital sign information over a period of time that may be sufficient to detect clinically relevant signs of the patient having an occurrence or recurrence of a disease or disorder. For the example, the patient who has been treated for a disease or disorder at a hospital or other clinical setting may need to be monitored for occurrence or recurrence of the disease or disorder (or occurrence of a complication related to an administered treatment for the disease or disorder). For example, a patient who has received an operation (e.g., a surgery such as an organ transplant) may need to be monitored for an occurrence of sepsis or other post-operative complications related to the operation (e.g., post-surgery complications). Patient monitoring may include detecting conditions that cause sepsis (e.g., bacteria or virus). Patient monitoring may detect complications such as stroke, pneumonia, heart failure, myocardial infarction (heart attack), chronic obstructive pulmonary disease (COPD), general deterioration, influenza, atrial fibrillation, and panic or anxiety attack. Such patient monitoring may be performed in a hospital or other clinical setting using specialized equipment such as medical monitors (e.g., cardiac monitoring, respiratory monitoring, neurological monitoring, blood glucose monitoring, hemodynamic monitoring, and body temperature monitoring) to measure and/or collect vital sign information (e.g., heart rate, blood pressure, respiratory rate, and pulse oximetry). However, patient monitoring outside of a clinical setting (e.g., a hospital) may pose challenges for non-invasive collection of vital sign information and accurate prediction of occurrence or recurrence of a disease or disorder.

Recognized herein is the need for systems and methods for patient monitoring by continuous collection and analysis of vital sign information. Such analysis of vital sign information (e.g., heart rate and/or blood pressure) of a subject (patient) may be performed by a wearable monitoring device (e.g., at the subject's home, instead of a clinical setting such as a hospital) over a period of time to predict a likelihood of the subject having a disease or disorder (e.g., sepsis) or a complication related to an administered treatment for a disease or disorder.

The present disclosure provides systems and methods that may advantageously collect and analyze vital sign information from a subject over a period of time to accurately and non-invasively predict a likelihood of the subject having a disease or disorder (e.g., sepsis) or a complication related to an administered treatment for a disease or disorder. Such systems and methods may allow patients with elevated risk of a disease or disorder to be accurately monitored for recurrence outside of a clinical setting, thereby improving the accuracy of detection of occurrence or recurrence of a disease disorder, or complication; reducing clinical health care costs; and improving patients' quality of life. For example, such systems and methods may produce accurate detections or predictions of likelihood of occurrence or recurrence of a disease, disorder, or complication that are clinically actionable by physicians (or other health care workers) toward deciding whether to discharge patients from a hospital for monitoring in a home setting, thereby reducing clinical health care costs. As another example, such systems and methods may enable in-home patient monitoring, thereby increasing patients' quality of life compared to remaining hospitalized or making frequent visits to clinical care sites. A goal of patient monitoring (e.g., in-home) may include preventing hospital re-admissions for a discharged patient.

The collected and transmitted vital sign information may be aggregated, for example, by batching and uploading to a computer server (e.g., a secure cloud database), where artificially intelligent algorithms may analyze the data in a continuous or real-time manner. If an adverse health condition (e.g., deterioration of the patient's state, occurrence or recurrence of a disease or disorder, or occurrence of a complication) is detected or predicted, the computer server may send a real-time alert to a health care provider (e.g., a general practitioner and/or treating physician). The health care provider may subsequently perform follow-up care, such as contacting the patient and requesting that the patient return to the hospital for further treatment or clinical inspection (e.g., monitoring, diagnosis, or prognosis). Alternatively or in combination, the health care provider may prescribe a treatment or a clinical procedure to be administered to the patient based on the real-time alert.

### Monitoring System Overview

A monitoring system may be used to collect and analyze vital sign information from a subject over a period of time to predict a likelihood of the subject having a disease, disorder, or complication related to an administered treatment for a disease or disorder. The monitoring system may comprise a wearable monitoring device. For example, the wearable monitoring device may be attached to a subject's chest and collect and transmit vital sign information to the subject's smartphone or other mobile device. The monitoring system may be used in a hospital or other clinical setting or in a home setting of the subject.

The monitoring system may comprise a wearable monitoring device (e.g., an electronic device or a monitoring patch), a mobile phone application, a database, and an artificial intelligence-based analytics engine to prevent hospital admission and re-admission in a user (e.g., a chronically ill patient) by detecting or predicting an adverse health condition (e.g., deterioration of the patient's state, occurrence or recurrence of a disease or disorder, or occurrence of a complication) in the user.

The wearable monitoring device (e.g., an electronic device or a monitoring patch) may be configured to measure, collect, and/or record health data, such as vital sign data comprising physiological signals (e.g., heart rate, respiration rate, and heart-rate variability) from the user's body (e.g., at the torso). The wearable monitoring device may be further configured to transmit such vital sign data (e.g., wirelessly) to a mobile device of the user (e.g., a smartphone, a tablet, a laptop, a smart watch, or smart glasses). Examples of vital sign data may include heart rate, heart rate variability, blood pressure, respiratory rate, blood oxygen concentration (e.g., by pulse oximetry), carbon dioxide concentration in respiratory gases, a hormone level, sweat analysis, blood glucose, body temperature, impedance (e.g., bioimpedance), conductivity, capacitance, resistivity, electromyography, galvanic skin response, neurological signals (e.g., electroencephalography), and immunology markers. The data may be measured, collected, and/or recorded in real-time (e.g., by using suitable biosensors and/or mechanical sensors), and may be transmitted continuously to the mobile device (e.g., through a wireless transceiver such as a Bluetooth transceiver or cellular radio transceiver (e.g., 3G, 4G, LTE, or 5G)). In some embodiments, the wearable monitoring device may transmit the data directly (e.g., to a computer, server, or distributed network) using a cellular radio transceiver (e.g., 3G, 4G, LTE, or 5G). The device may be used to monitor a subject (e.g., patient) over a period of time based on the acquired health data, for example, by detecting or predicting an adverse health condition (e.g., deterioration of the patient's state, occurrence or recurrence of a disease or disorder, or occurrence of a complication) of the subject over the period of time.

The mobile application may be configured to allow a user to pair with, control, and view data from the wearable monitoring device. For example, the mobile application may be configured to allow a user to use a mobile device (e.g., a smartphone, a tablet, a laptop, a smart watch, or smart glasses) to pair with the wearable monitoring device (e.g., through a wireless transceiver such as a Bluetooth transceiver or cellular radio transceiver (e.g., 3G, 4G, LTE, or 5G)) for transmission of data and/or control signals. In some embodiments, the wearable monitoring device may transmit the data directly (e.g., to a computer, server, or distributed network) using a cellular radio transceiver (e.g., 3G, 4G, LTE, or 5G). The mobile application may comprise a graphical user interface (GUI) to allow the user to view trends, statistics, and/or alerts generated based on their measured, collected, or recorded vital sign data (e.g., currently measured data, previously collected or recorded data, or a combination thereof). For example, the GUI may allow the user to view historical or average trends of a set of vital sign data over a period of time (e.g., on an hourly basis, on a daily basis, on a weekly basis, or on a monthly basis). The mobile application may further communicate with a web-based software application, which may be configured to store and analyze the recorded vital sign data. For example, the recorded vital sign data may be stored in a database (e.g., a computer server or on a cloud network) for real-time or future processing and analysis.

Health care providers, such as physicians and treating teams of a patient (e.g., the user) may have access to patient alerts, data (e.g., vital sign data), and/or predictions or assessments generated from such data. Such access may be provided by a web-based dashboard (e.g., a GUI). The web-based dashboard may be configured to display, for example, patient metrics, recent alerts, and/or prediction of health outcomes (e.g., rate or likelihood of deterioration and/or sepsis). Using the web-based dashboard, health care providers may determine clinical decisions or outcomes based at least in part on such displayed alerts, data, and/or predictions or assessments generated from such data.

For example, a physician may instruct the patient to undergo one or more clinical tests at the hospital or other clinical site, based at least in part on patient metrics or on alerts detecting or predicting an adverse health condition (e.g., deterioration of the patient's state, occurrence or recurrence of a disease or disorder, or occurrence of a complication) of the subject over a period of time. The monitoring system may generate and transmit such alerts to health care providers when a certain predetermined criterion is met (e.g., a minimum threshold for a likelihood of deterioration of the patient's state, occurrence or recurrence of a disease or disorder, or occurrence of a complication such as sepsis).

Such a minimum threshold may be, for example, at least about a 5% likelihood, at least about a 10% likelihood, at least about a 20% likelihood, at least about a 25% likelihood, at least about a 30% likelihood, at least about a 35% likelihood, at least about a 40% likelihood, at least about a 45% likelihood, at least about a 50% likelihood, at least about a 55% likelihood, at least about a 60% likelihood, at least about a 65% likelihood, at least about a 70% likelihood, at least about a 75% likelihood, at least about an 80% likelihood, at least about a 85% likelihood, at least about a 90% likelihood, at least about a 95% likelihood, at least about a 96% likelihood, at least about a 97% likelihood, at least about a 98% likelihood, or at least about a 99% likelihood.

As another example, a physician may prescribe a therapeutically effective dose of a treatment (e.g., drug), a clinical procedure, or further clinical testing to be administered to the patient based at least in part on patient metrics or on alerts detecting or predicting an adverse health condition (e.g., sepsis, deterioration of the patient's state, occurrence or recurrence of a disease or disorder, or occurrence of a complication) of the subject over a period of time. For example, the physician may prescribe an anti-inflammatory therapeutic in response to an indication of inflammation in the patient, or an analgesic therapeutic in response to an indication of pain in the patient. Such a prescription of a therapeutically effective dose of a treatment (e.g., drug), a clinical procedure, or further clinical testing may be determined without requiring an in-person clinical appointment with the prescribing physician. The physician may prescribe an anti-microbial therapy (e.g., to treat sepsis in a patient), such as orally administered broad-spectrum antibiotics (e.g., ciprofloxacin, amoxicillin, norfloxacin, Aminoglycosides, Carbapenems, Augmentin, other Cephlasporins, etc.). Oral broad-spectrum antibiotics may target gram-negative bacteria because of their higher death rates in response to treatment. In some cases, oral antimicrobial treatment may be ineffective or sub-optimally effective, and a patient may receive intravenous (IV) antibiotics in a hospital or other clinical setting.

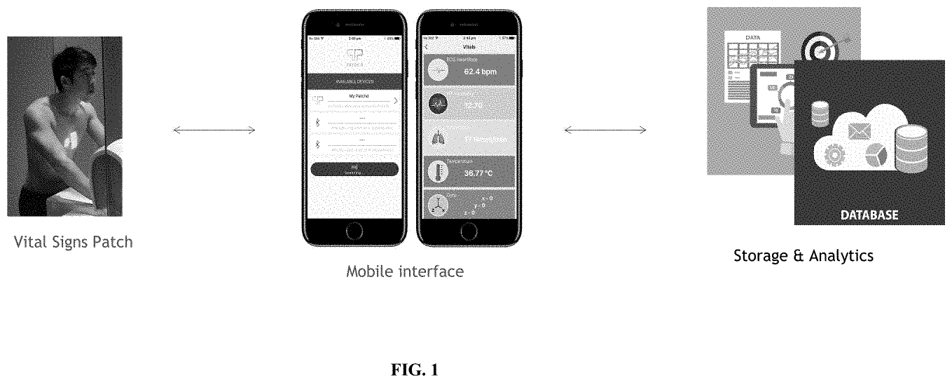

An overview of the system architecture is illustrated in FIG. 1. The system may comprise a wearable monitoring device, a mobile device application, and a web database. The system may comprise a vital signs device (e.g., a wearable monitoring device to measure health data of a patient), a mobile interface (e.g., graphical user interface, or GUI) of the mobile device application (e.g., to enable a user to control collection, measurement, recording, storage, and/or analysis of health data for prediction of health outcomes), and computer hardware and/or software for storage and/or analytics of the collected health data (e.g., vital sign information).

The mobile device application of the monitoring system may utilize or access external capabilities of artificial intelligence techniques to develop signatures for patient deterioration and disease states. The web-based software may further use these signatures to accurately predict deterioration (e.g., hours to days earlier than with traditional clinical care). Using such a predictive capability, health care providers (e.g., physicians) may be able to make informed, accurate risk-based decisions, thereby allowing more at-risk patients to be treated from home.

The mobile device application may analyze acquired health data from a subject (patient) to generate a likelihood of the subject having an adverse health condition (e.g., deterioration of the patient's state, occurrence or recurrence of a disease or disorder, or occurrence of a complication). For example, the mobile device application may apply a trained (e.g., prediction) algorithm to the acquired health data to generate the likelihood of the subject having an adverse health condition (e.g., deterioration of the patient's state, occurrence or recurrence of a disease or disorder, or occurrence of a complication). The trained algorithm may comprise an artificial intelligence based classifier, such as a machine learning based classifier, configured to process the acquired health data to generate the likelihood of the subject having the disease or disorder. The machine learning classifier may be trained using clinical datasets from one or more cohorts of patients, e.g., using clinical health data of the patients (e.g., vital sign data) as inputs and known clinical health outcomes (e.g., occurrence or recurrence of a disease or disorder) of the patients as outputs to the machine learning classifier. The trained algorithm may be configured to identify the adverse health condition with an accuracy of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more than 99%, for at least about 100, at least about 500, at least about 1,000, at least about 5,000, at least about 10,000, at least about 30,000, or more than about 30,000 independent samples.

The machine learning classifier may comprise one or more machine learning algorithms. Examples of machine learning algorithms may include a support vector machine (SVM), a naïve Bayes classification, a random forest, a neural network (such as a deep neural network (DNN), a recurrent neural network (RNN), a deep RNN, a long short-term memory (LSTM) recurrent neural network (RNN), or a gated recurrent unit (GRU) recurrent neural network (RNN)), deep learning, or other supervised learning algorithm or unsupervised learning algorithm for classification and regression. The machine learning classifier may be trained using one or more training datasets corresponding to patient data.

The trained algorithm may be configured to accept a plurality of input variables and to produce one or more output values based on the plurality of input variables. The plurality of input variables may comprise one or more datasets indicative of an adverse health condition. For example, input variables may comprise vital sign measurements of a subject. The plurality of input variables may also include clinical health data of a subject.

Training datasets may be generated from, for example, one or more cohorts of patients having common clinical characteristics (features) and clinical outcomes (labels). Training datasets may comprise a set of features and labels corresponding to the features. Features may correspond to algorithm inputs comprising patient demographic information derived from electronic medical records (EMR) and medical observations. Features may comprise clinical characteristics such as, for example, certain ranges or categories of vital sign measurements, such as heart rate, heart rate variability, blood pressure (e.g., systolic and diastolic), respiratory rate, blood oxygen concentration (SpO₂), carbon dioxide concentration in respiratory gases, a hormone level, sweat analysis, blood glucose, body temperature, impedance (e.g., bioimpedance), conductivity, capacitance, resistivity, electromyography, galvanic skin response, neurological signals (e.g., electroencephalography), immunology markers, and other physiological measurements. Features may comprise patient information such as patient age, patient medical history, other medical conditions, current or past medications, and time since the last observation. For example, a set of features collected from a given patient at a given time point may collectively serve as a vital sign signature, which may be indicative of a health state or status of the patient at the given time point.

For example, ranges of vital sign measurements may be expressed as a plurality of disjoint continuous ranges of continuous measurement values, and categories of vital sign measurements may be expressed as a plurality of disjoint sets of measurement values (e.g., {“high”, “low”}, {“high”, {“normal”}, “normal”}, {“high”, “borderline high”, “normal”, “low”}, etc.). Clinical characteristics may also include clinical labels indicating the patient's health history, such as a diagnosis of a disease or disorder, a previous administration of a clinical treatment (e.g., a drug, a surgical treatment, chemotherapy, radiotherapy, immunotherapy, etc.), behavioral factors, or other health status (e.g., hypertension or high blood pressure, hyperglycemia or high blood glucose, hypercholesterolemia or high blood cholesterol, history of allergic reaction or other adverse reaction, etc.).

Labels may comprise clinical outcomes such as, for example, a presence, absence, diagnosis, or prognosis of an adverse health condition (e.g., deterioration of the patient's state, occurrence or recurrence of a disease or disorder, or occurrence of a complication) in the patient. Clinical outcomes may include a temporal characteristic associated with the presence, absence, diagnosis, or prognosis of the adverse health condition in the patient. For example, temporal characteristics may be indicative of the patient having had an occurrence of the adverse health condition (e.g., sepsis) within a certain period of time after a previous clinical outcome (e.g., being discharged from the hospital, undergoing an organ transplantation or other surgical operation, undergoing a clinical procedure, etc.). Such a period of time may be, for example, about 1 hour, about 2 hours, about 3 hours, about 4 hours, about 6 hours, about 8 hours, about 10 hours, about 12 hours, about 14 hours, about 16 hours, about 18 hours, about 20 hours, about 22 hours, about 24 hours, about 2 days, about 3 days, about 4 days, about 5 days, about 6 days, about 7 days, about 10 days, about 2 weeks, about 3 weeks, about 4 weeks, about 1 month, about 2 months, about 3 months, about 4 months, about 6 months, about 8 months, about 10 months, about 1 year, or more than about 1 year.

Input features may be structured by aggregating the data into bins or alternatively using a one-hot encoding with the time since the last observation included. Inputs may also include feature values or vectors derived from the previously mentioned inputs, such as cross-correlations calculated between separate vital sign measurements over a fixed period of time, and the discrete derivative or the finite difference between successive measurements. Such a period of time may be, for example, about 1 hour, about 2 hours, about 3 hours, about 4 hours, about 6 hours, about 8 hours, about 10 hours, about 12 hours, about 14 hours, about 16 hours, about 18 hours, about 20 hours, about 22 hours, about 24 hours, about 2 days, about 3 days, about 4 days, about 5 days, about 6 days, about 7 days, about 10 days, about 2 weeks, about 3 weeks, about 4 weeks, about 1 month, about 2 months, about 3 months, about 4 months, about 6 months, about 8 months, about 10 months, about 1 year, or more than about 1 year.

Training records may be constructed from sequences of observations. Such sequences may comprise a fixed length for ease of data processing. For example, sequences may be zero-padded or selected as independent subsets of a single patient's records.

The machine learning classifier algorithm may process the input features to generate output values comprising one or more classifications, one or more predictions, or a combination thereof. For example, such classifications or predictions may include a binary classification of a disease or a non-disease state, a classification between a group of categorical labels (e.g., ‘no sepsis’, ‘sepsis apparent’, and ‘sepsis likely’), a likelihood (e.g., relative likelihood or probability) of developing a particular disease or disorder (e.g., sepsis), a score indicative of a ‘presence of infection’, a score indicative of a level of systemic inflammation experienced by the patient, a ‘risk factor’ for the likelihood of mortality of the patient, a prediction of the time at which the patient is expected to have developed the disease or disorder, and a confidence interval for any numeric predictions. Various machine learning techniques may be cascaded such that the output of a machine learning technique may also be used as input features to subsequent layers or subsections of the machine learning classifier.

In some embodiments, some of the output values of the machine learning classifier may comprise numerical values, such as binary, integer, or continuous values. Such binary output values may comprise, for example, {0, 1}, {positive, negative}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may indicate a diagnosis or a prognosis of the adverse health condition of the subject. Some numerical values may be mapped to descriptive labels, for example, by mapping 1 to “positive” and 0 to “negative.”

Some of the output values may be assigned based on one or more cutoff values. For example, a binary classification of a subject may assign an output value of “positive” or 1 if the subject's vital sign data indicates that the subject has at least a 50% probability of having an adverse health condition. For example, a binary classification of a subject may assign an output value of “negative” or 0 if the subject's vital sign data indicates that the subject has less than a 50% probability of having an adverse health condition. In this case, a single cutoff value or classification threshold of 50% is used to classify subject's vital sign data into one of the two possible binary output values. Examples of single cutoff values or classification thresholds may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, a classification of a subject's vital sign data may assign an output value of “positive” or 1 if the subject's vital sign data indicates that the subject has a probability of having an adverse health condition of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of a subject's vital sign data may assign an output value of “positive” or 1 if the subject's vital sign data indicates that the subject has a probability of having an adverse health condition of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classification of a subject's vital sign data may assign an output value of “negative” or 0 if the subject's vital sign data indicates that the subject has a probability of an adverse health condition of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of the subject's vital sign data may assign an output value of “negative” or 0 if the subject's vital sign data indicates that the subject has a probability of having an adverse health condition of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

The classification of the subject's vital sign data may assign an output value of “indeterminate” or 2 if the subject's vital sign data is not classified as “positive”, “negative”, 1, or 0. In this case, a set of two cutoff values or classification thresholds is used to classify the subject's vital sign data into one of the three possible output values. Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values or classification thresholds may be used to classify the subject's vital sign data into one of n+1 possible output values, where n is any positive integer.

In order to train the machine learning classifier model (e.g., by determining weights and correlations of the model) to generate real-time classifications or predictions, the model can be trained using datasets. Such datasets may be sufficiently large to generate statistically significant classifications or predictions. For example, datasets may comprise: intensive care unit (ICU) databases of de-identified data including vital sign observations (e.g., labeled with an appearance of ICD9 or ICD10 diagnosis codes), databases of ambulatory vital sign observations collected via tele-health programs, databases of vital sign observations collected from rural communities, vital sign observations collected from fitness trackers, vital sign observations from a hospital or other clinical setting, vital sign measurements collected using an FDA-approved wearable monitoring device, and vital sign measurements collected using wearable monitoring devices of the present disclosure.

Examples of databases include open source databases such as MIMIC-III (Medical Information Mart for Intensive Care III) and the eICU Collaborative Research Database (Philips). The MIMIC III database may comprise de-identified patient records, vital sign measurements, laboratory test results, procedures, and medications prescribed at the Beth Israel Deaconess Medical Center from the time period between 2001 and 2012. The Philips eICU program is a critical care tele-health program providing supplementary information to remote caregivers in the intensive care unit. Datasets from the eICU Collaborative Research Database may comprise de-identified information derived from vital sign measurements, patient demographics, and medications and treatments captured within the system. In contrast to the MIMIC III database, the eICU database may contain data collected from multiple different hospitals, rather than a single hospital.

In some cases, datasets are annotated or labeled. For example, to identify and label the onset of sepsis in training records, methods involving the definitions of sepsis-2 or sepsis-3 may be used.

The trained algorithm may be trained with a plurality of independent training samples. Each of the independent training samples may comprise a set of vital sign data and/or clinical characteristics obtained from a subject, and one or more known output values corresponding to the subject (e.g., a clinical diagnosis, prognosis, absence, or treatment efficacy of an adverse health condition of the subject). Independent training samples may comprise sets of vital sign data and/or clinical characteristics and associated outputs obtained or derived from a plurality of different subjects. Independent training samples may comprise sets of vital sign data and/or clinical characteristics and associated outputs obtained at a plurality of different time points from the same subject (e.g., on a regular basis such as weekly, biweekly, or monthly). Independent training samples may be associated with presence of the adverse health condition (e.g., training samples comprising sets of vital sign data and/or clinical characteristics and associated outputs obtained or derived from a plurality of subjects known to have the adverse health condition). Independent training samples may be associated with absence of the adverse health condition (e.g., training samples comprising sets of vital sign data and/or clinical characteristics and associated outputs obtained or derived from a plurality of subjects who are known to not have a previous diagnosis of the adverse health condition, who are asymptomatic for the adverse health condition, or who have received a negative test result for the adverse health condition).

The trained algorithm may be trained with at least about 100, at least about 500, at least about 1,000, at least about 5,000, at least about 10,000, at least about 20,000, at least about 30,000, at least about 35,000, at least about 40,000, at least about 45,000, at least about 50,000, or more than about 50,000 independent training samples. The independent training samples may comprise samples associated with a presence of an adverse health condition and samples associated with an absence of the adverse health condition.

The trained algorithm may be trained with a first number of independent training samples associated with a presence of an adverse health condition and a second number of independent training samples associated with an absence of the adverse health condition. The first number of independent training samples associated with a presence of the adverse health condition may be no more than the second number of independent training samples associated with an absence of the adverse health condition. The first number of independent training samples associated with a presence of the adverse health condition may be equal to the second number of independent training samples associated with an absence of the adverse health condition. The first number of independent training samples associated with a presence of the adverse health condition may be greater than the second number of independent training samples associated with an absence of the adverse health condition.

Datasets may be split into subsets (e.g., discrete or overlapping), such as a training dataset, a development dataset, and a test dataset. For example, a dataset may be split into a training dataset comprising 80% of the dataset, a development dataset comprising 10% of the dataset, and a test dataset comprising 10% of the dataset. The training dataset may comprise about 10%, about 20%, about 30%, about 40%, about 50%, about 60%, about 70%, about 80%, or about 90% of the dataset. The development dataset may comprise about 10%, about 20%, about 30%, about 40%, about 50%, about 60%, about 70%, about 80%, or about 90% of the dataset. The test dataset may comprise about 10%, about 20%, about 30%, about 40%, about 50%, about 60%, about 70%, about 80%, or about 90% of the dataset. Training sets (e.g., training datasets) may be selected by random sampling of a set of data corresponding to one or more patient cohorts to ensure independence of sampling. Alternatively, training sets (e.g., training datasets) may be selected by proportionate sampling of a set of data corresponding to one or more patient cohorts to ensure independence of sampling.

To improve the accuracy of model predictions and reduce overfitting of the model, the datasets may be augmented to increase the number of samples within the training set. For example, data augmentation may comprise rearranging the order of observations in a training record. To accommodate datasets having missing observations, methods to impute missing data may be used, such as forward-filling, back-filling, linear interpolation, and multi-task Gaussian processes. Datasets may be filtered to remove confounding factors. For example, within ICU databases, patients that have repeated events of septic infections may be excluded.

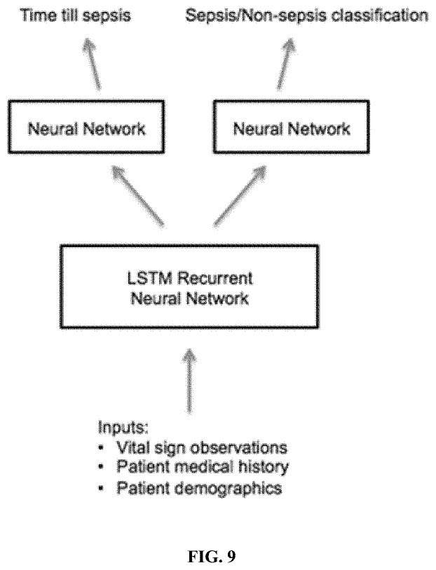

The machine learning classifier may comprise one or more neural networks, such as a deep neural network (DNN), a recurrent neural network (RNN), or a deep RNN. The recurrent neural network may comprise units which can be long short-term memory (LSTM) units or gated recurrent units (GRU). For example, as shown in FIG. 9, the machine learning classifier may comprise an algorithm architecture comprising a long short-term memory (LSTM) recurrent neural network (RNN), with a set of input features such as vital sign observations, patient medical history, and patient demographics. Neural network techniques, such as dropout or regularization, may be used during training the machine learning classifier to prevent overfitting.

The trained algorithm may be configured to identify the adverse health condition at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The accuracy of identifying the adverse health condition by the trained algorithm may be calculated as the percentage of independent test samples (e.g., subjects known to have the adverse health condition or subjects with negative clinical test results for the adverse health condition) that are correctly identified or classified as having or not having the adverse health condition.

The trained algorithm may be configured to identify the adverse health condition with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the adverse health condition using the trained algorithm may be calculated as the percentage of samples identified or classified as having the adverse health condition that correspond to subjects that truly have the adverse health condition.

The trained algorithm may be configured to identify the adverse health condition with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the adverse health condition using the trained algorithm may be calculated as the percentage of samples identified or classified as not having the adverse health condition that correspond to subjects that truly do not have the adverse health condition.

The trained algorithm may be configured to identify the adverse health condition with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the adverse health condition using the trained algorithm may be calculated as the percentage of independent test samples associated with presence of the adverse health condition (e.g., subjects known to have the adverse health condition) that are correctly identified or classified as having the adverse health condition.

The trained algorithm may be configured to identify the adverse health condition with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the adverse health condition using the trained algorithm may be calculated as the percentage of independent test samples associated with absence of the adverse health condition (e.g., subjects with negative clinical test results for the adverse health condition) that are correctly identified or classified as not having the adverse health condition.

The trained algorithm may be configured to identify the adverse health condition with an Area Under the Receiver Operating Characteristic (AUROC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUROC may be calculated as an integral of the Receiver Operator Characteristic (ROC) curve (e.g., the area under the ROC curve) associated with the trained algorithm in classifying samples as having or not having the adverse health condition.

The trained algorithm may be configured to identify the adverse health condition with an Area Under the Precision-Recall Curve (AUPRC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUPRC may be calculated as an integral of the precision-recall curve (e.g., the area under the precision-recall curve) associated with the trained algorithm in classifying samples as having or not having the adverse health condition.

The trained algorithm may be adjusted or tuned to improve one or more of the performance, accuracy, PPV, NPV, clinical sensitivity, clinical specificity, AUROC, or AUPRC of identifying the adverse health condition. The trained algorithm may be adjusted or tuned by adjusting parameters of the trained algorithm (e.g., a set of cutoff values or classification thresholds used to classify a sample as described elsewhere herein, or weights of a neural network). The trained algorithm may be adjusted or tuned continuously during the training process or after the training process has completed.

After the trained algorithm is initially trained, a subset of the inputs may be identified as most influential or most important to be included for making high-quality classifications. For example, a subset of a plurality of vital sign data (e.g., types of vital sign measurements) may be identified as most influential or most important to be included for making high-quality classifications or identifications of adverse health conditions. The plurality of vital sign data or a subset thereof may be ranked based on classification metrics indicative of each vital sign's influence or importance toward making high-quality classifications or identifications of adverse health conditions. Such metrics may be used to reduce, in some cases significantly, the number of input variables (e.g., predictor variables) that may be used to train the trained algorithm to a desired performance level (e.g., based on a desired minimum accuracy, PPV, NPV, clinical sensitivity, clinical specificity, AUROC, AUPRC, or a combination thereof). For example, if training the trained algorithm with a plurality comprising several dozen of input variables in the trained algorithm results in an accuracy of classification of more than 99%, then training the trained algorithm instead with only a selected subset of no more than about 50, no more than about 40, no more than about 30, no more than about 20, no more than about 15, no more than 14, no more than 13, no more than 12, no more than 11, no more than 10, no more than 9, no more than 8, no more than 7, no more than 6, no more than 5, no more than 4, no more than 3, no more than 2, or no more than 1 such most influential or most important input variables among the plurality can yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The subset may be selected by rank-ordering the entire plurality of input variables and selecting a predetermined number (e.g., no more than about 50, no more than about 40, no more than about 30, no more than about 20, no more than about 15, no more than 14, no more than 13, no more than 12, no more than 11, no more than 10, no more than 9, no more than 8, no more than 7, no more than 6, no more than 5, no more than 4, no more than 3, no more than 2, or no more than 1) of input variables with the best classification metrics.

The adverse health condition of the subject may be monitored, e.g., by monitoring a course of treatment for treating the adverse health condition of the subject. The monitoring may comprise assessing the adverse health condition of the subject at two or more time points. The assessing may be based at least on the assessments generated by the machine learning classifier based on input vital sign data obtained at each of the two or more time points.

In some embodiments, a difference in the assessments of the machine learning classifier determined between the two or more time points may be indicative of one or more clinical indications, such as (i) a diagnosis of the adverse health condition of the subject, (ii) a prognosis of the adverse health condition of the subject, (iii) an increased risk of the adverse health condition of the subject, (iv) a decreased risk of the adverse health condition of the subject, (v) an efficacy of the course of treatment for treating the adverse health condition of the subject, and (vi) a non-efficacy of the course of treatment for treating the adverse health condition of the subject.

In some embodiments, a difference in the assessments of the machine learning classifier determined between the two or more time points may be indicative of a diagnosis of the adverse health condition of the subject. For example, if the adverse health condition was not detected in the subject at an earlier time point but was detected in the subject at a later time point, then the difference is indicative of a diagnosis of the adverse health condition of the subject. A clinical action or decision may be made based on this indication of diagnosis of the adverse health condition of the subject, such as, for example, prescribing a new therapeutic intervention for the subject. The clinical action or decision may comprise recommending the subject for a secondary clinical test to confirm the diagnosis of the adverse health condition. This secondary clinical test may comprise an imaging test, a blood test, or any combination thereof.

In some embodiments, a difference in the assessments of the machine learning classifier determined between the two or more time points may be indicative of a prognosis of the adverse health condition of the subject.

In some embodiments, a difference in the assessments of the machine learning classifier determined between the two or more time points may be indicative of the subject having an increased risk of the adverse health condition. For example, if the adverse health condition was detected in the subject both at an earlier time point and at a later time point, and if the difference is a negative difference (e.g., the assessments of the machine learning classifier increased from the earlier time point to the later time point), then the difference may be indicative of the subject having an increased risk of the adverse health condition. A clinical action or decision may be made based on this indication of the increased risk of the adverse health condition, e.g., prescribing a new therapeutic intervention or switching therapeutic interventions (e.g., ending a current treatment and prescribing a new treatment) for the subject. The clinical action or decision may comprise recommending the subject for a secondary clinical test to confirm the increased risk of the adverse health condition. This secondary clinical test may comprise an imaging test, a blood test, or any combination thereof.

In some embodiments, a difference in the assessments of the machine learning classifier determined between the two or more time points may be indicative of the subject having a decreased risk of the adverse health condition. For example, if the adverse health condition was detected in the subject both at an earlier time point and at a later time point, and if the difference is a positive difference (e.g., the assessments of the machine learning classifier decreased from the earlier time point to the later time point), then the difference may be indicative of the subject having a decreased risk of the adverse health condition. A clinical action or decision may be made based on this indication of the decreased risk of the adverse health condition (e.g., continuing or ending a current therapeutic intervention) for the subject. The clinical action or decision may comprise recommending the subject for a secondary clinical test to confirm the decreased risk of the adverse health condition. This secondary clinical test may comprise an imaging test, a blood test, or any combination thereof.

In some embodiments, a difference in the assessments of the machine learning classifier determined between the two or more time points may be indicative of an efficacy of the course of treatment for treating the adverse health condition of the subject. For example, if the adverse health condition was detected in the subject at an earlier time point but was not detected in the subject at a later time point, then the difference may be indicative of an efficacy of the course of treatment for treating the adverse health condition of the subject. A clinical action or decision may be made based on this indication of the efficacy of the course of treatment for treating the adverse health condition of the subject, e.g., continuing or ending a current therapeutic intervention for the subject. The clinical action or decision may comprise recommending the subject for a secondary clinical test to confirm the efficacy of the course of treatment for treating the adverse health condition. This secondary clinical test may comprise an imaging test, a blood test, or any combination thereof.

In some embodiments, a difference in the assessments of the machine learning classifier determined between the two or more time points may be indicative of a non-efficacy of the course of treatment for treating the adverse health condition of the subject. For example, if the adverse health condition was detected in the subject both at an earlier time point and at a later time point, and if the difference is a negative or zero difference (e.g., the assessments of the machine learning classifier increased or remained at a constant level from the earlier time point to the later time point), and if an efficacious treatment was indicated at an earlier time point, then the difference may be indicative of a non-efficacy of the course of treatment for treating the adverse health condition of the subject. A clinical action or decision may be made based on this indication of the non-efficacy of the course of treatment for treating the adverse health condition of the subject, e.g., ending a current therapeutic intervention and/or switching to (e.g., prescribing) a different new therapeutic intervention for the subject. The clinical action or decision may comprise recommending the subject for a secondary clinical test to confirm the non-efficacy of the course of treatment for treating the adverse health condition. This secondary clinical test may comprise an imaging test, a blood test, or any combination thereof.

When the machine learning classifier generates a classification or a prediction of a disease, disorder, or complication, an alert or alarm may be generated and transmitted to a health care provider, such as a physician, nurse, or other member of the patient's treating team within a hospital. Alerts may be transmitted via an automated phone call, a short message service (SMS) or multimedia message service (MMS) message, an e-mail, or an alert within a dashboard. The alert may comprise output information such as a prediction of a disease, disorder, or complication, a likelihood of the predicted disease, disorder, or complication, a time until an expected onset of the disease, disorder, or condition, a confidence interval of the likelihood or time, or a recommended course of treatment for the disease, disorder, or complication. As shown in FIG. 9, the LSTM recurrent neural network may comprise a plurality of sub-networks, each of which is configured to generate a classification or prediction of a different type of output information (e.g., a sepsis/non-sepsis classification and a time until the onset of sepsis).

To validate the performance of the machine learning classifier model, different performance metrics may be generated. For example, an area under the receiver-operating curve (AUROC) may be used to determine the diagnostic capability of the machine learning classifier. For example, the machine learning classifier may use classification thresholds which are adjustable, such that specificity and sensitivity are tunable, and the receiver-operating curve (ROC) can be used to identify the different operating points corresponding to different values of specificity and sensitivity.

In some cases, such as when datasets are not sufficiently large, cross-validation may be performed to assess the robustness of a machine learning classifier model across different training and testing datasets.

In some cases, while a machine learning classifier model may be trained using a dataset of records which are a subset of a single patient's observations, the performance of the classifier model's discrimination ability (e.g., as assessed using an AUROC) is calculated using the entire record for a patient. To calculate performance metrics such as sensitivity, specificity, accuracy, positive predictive value (PPV), negative predictive value (NPV), AUPRC, AUROC, or similar, the following definitions may be used. A “false positive” may refer to an outcome in which if an alert or alarm has been incorrectly or prematurely activated (e.g., before the actual onset of, or without any onset of, a disease state or condition such as sepsis) fires too early. A “true positive” may refer to an outcome in which an alert or alarm has been activated at the correct time (within a predetermined buffer or tolerance), and the patient's record indicates the disease or condition (e.g., sepsis). A “false negative” may refer to an outcome in which no alert or alarm has been activated, but the patient's record indicates the disease or condition (e.g., sepsis). A “true negative” may refer to an outcome in which no alert or alarm has been activated, and the patient's record does not indicate the disease or condition (e.g., sepsis).

The machine learning classifier may be trained until certain predetermined conditions for accuracy or performance are satisfied, such as having minimum desired values corresponding to diagnostic accuracy measures. For example, the diagnostic accuracy measure may correspond to prediction of a likelihood of occurrence of an adverse health condition such as deterioration or a disease or disorder (e.g., sepsis) of the subject. As another example, the diagnostic accuracy measure may correspond to prediction of a likelihood of deterioration or recurrence of an adverse health condition such as a disease or disorder for which the subject has previously been treated. For example, a diagnostic accuracy measure may correspond to prediction of likelihood of recurrence of an infection in a subject who has previously been treated for the infection. Examples of diagnostic accuracy measures may include sensitivity, specificity, positive predictive value (PPV), negative predictive value (NPV), accuracy, area under the precision-recall curve (AUPRC), and area under the curve (AUC) of a Receiver Operating Characteristic (ROC) curve (AUROC) corresponding to the diagnostic accuracy of detecting or predicting an adverse health condition.

For example, such a predetermined condition may be that the sensitivity of predicting occurrence or recurrence of the adverse health condition such as deterioration or a disease or disorder (e.g., onset of sepsis) comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a predetermined condition may be that the specificity of predicting occurrence or recurrence of the adverse health condition such as deterioration or a disease or disorder (e.g., onset of sepsis) comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a predetermined condition may be that the positive predictive value (PPV) of predicting occurrence or recurrence of the adverse health condition such as deterioration or a disease or disorder comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a predetermined condition may be that the negative predictive value (NPV) of predicting occurrence or recurrence of the adverse health condition such as deterioration or a disease or disorder (e.g., onset of sepsis) comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a predetermined condition may be that the area under the curve (AUC) of a Receiver Operating Characteristic (ROC) curve (AUROC) of predicting occurrence or recurrence of the adverse health condition such as deterioration or a disease or disorder (e.g., onset of sepsis) comprises a value of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

As another example, such a predetermined condition may be that the area under the precision-recall curve (AUPRC) of predicting occurrence or recurrence of the adverse health condition such as deterioration or a disease or disorder (e.g., onset of sepsis) comprises a value of at least about 0.10, at least about 0.15, at least about 0.20, at least about 0.25, at least about 0.30, at least about 0.35, at least about 0.40, at least about 0.45, at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

In some embodiments, the trained classifier may be trained or configured to predict occurrence or recurrence of the adverse health condition such as deterioration or a disease or disorder (e.g., onset of sepsis) with a sensitivity of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained classifier may be trained or configured to predict occurrence or recurrence of the adverse health condition such as deterioration or a disease or disorder (e.g., onset of sepsis) with a specificity of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained classifier may be trained or configured to predict occurrence or recurrence of the adverse health condition such as deterioration or a disease or disorder (e.g., onset of sepsis) with a positive predictive value (PPV) of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained classifier may be trained or configured to predict occurrence or recurrence of the adverse health condition such as deterioration or a disease or disorder (e.g., onset of sepsis) with a negative predictive value (NPV) of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained classifier may be trained or configured to predict occurrence or recurrence of the adverse health condition such as deterioration or a disease or disorder (e.g., onset of sepsis) with an area under the curve (AUC) of a Receiver Operating Characteristic (ROC) curve (AUROC) of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

In some embodiments, the trained classifier may be trained or configured to predict occurrence or recurrence of the adverse health condition such as deterioration or a disease or disorder (e.g., onset of sepsis) with an area under the precision-recall curve (AUPRC) of at least about 0.10, at least about 0.15, at least about 0.20, at least about 0.25, at least about 0.30, at least about 0.35, at least about 0.40, at least about 0.45, at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

In some embodiments, the trained classifier may be trained or configured to predict occurrence or recurrence of the adverse health condition such as deterioration or a disease or disorder (e.g., onset of sepsis) over a period of time before the actual occurrence or recurrence of the adverse health condition (e.g., a period of time including a window beginning about 1 hour, about 2 hours, about 3 hours, about 4 hours, about 5 hours, about 6 hours, about 7 hours, about 8 hours, about 9 hours, about 10 hours, about 12 hours, about 14 hours, about 16 hours, about 18 hours, about 20 hours, about 22 hours, about 24 hours, about 36 hours, about 48 hours, about 72 hours, about 96 hours, about 120 hours, about 6 days, or about 7 days prior to the onset of the health condition, and ending at the onset of the health condition).

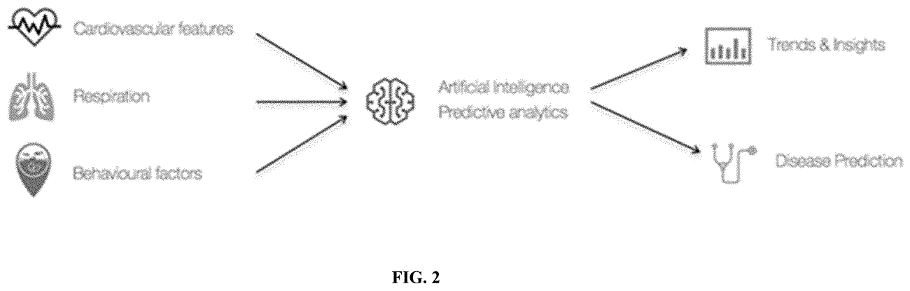

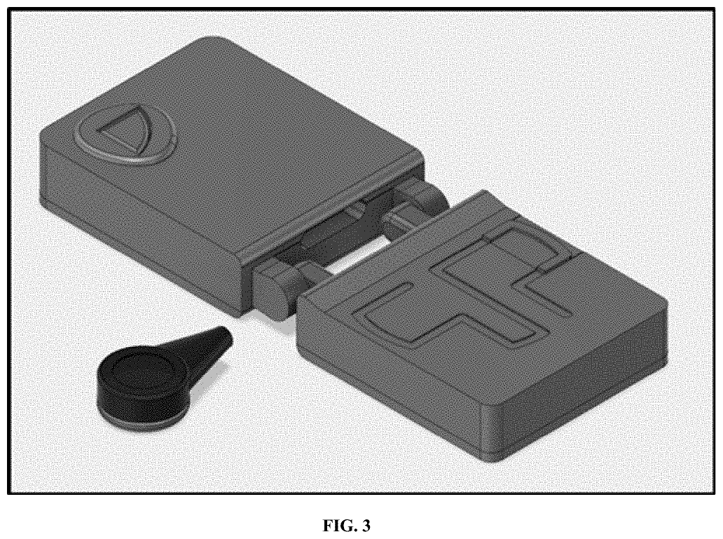

An example illustration of the data flows in the system architecture is shown in FIG. 2. Systems and methods provided herein may perform predictive analytics using artificial intelligence based approaches, by collecting and analyzing input data (e.g., cardiovascular features, respiration data, and behavioral factors) to yield output data (e.g., trends and insights into vital sign measurements, and predictions of adverse health conditions). Predictions of adverse health conditions may comprise, for example, a likelihood of the monitored subject having a disease or disorder (e.g., sepsis), or a likelihood of the monitored subject having deterioration or recurrence of a disease or disorder for which the subject has previously been treated.

### Design of Wearable Monitoring Device

The wearable monitoring device may be lightweight and discrete, and may comprise electronic sensors, a rechargeable lithium ion battery, electrode clips, and a physical enclosure. The electrode clips may comprise adhesive electrocardiogram (ECG) electrodes inserted therein, thereby allowing the device to reversibly attach to a patient's chest and measure ECG signals from the patient's skin. The wearable monitoring device may be configured to be worn under clothing and may be configured to be reversibly attachable to a patient's body and to operate (e.g., perform measurements of ECG signals) without requiring the patient's skin to be punctured or breached. For example, the wearable monitoring device may be reversibly attached to the patient's body (e.g., the torso or chest) using the adhesive ECG electrodes.

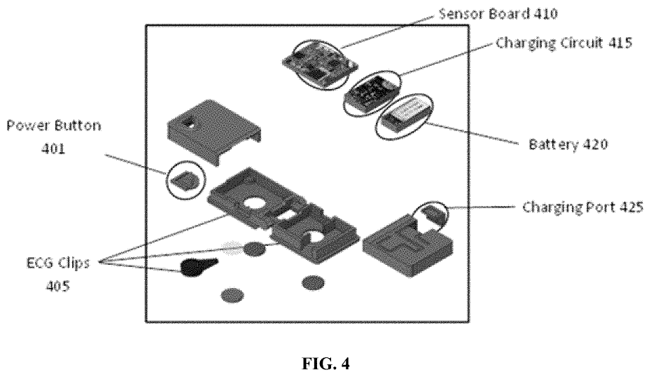

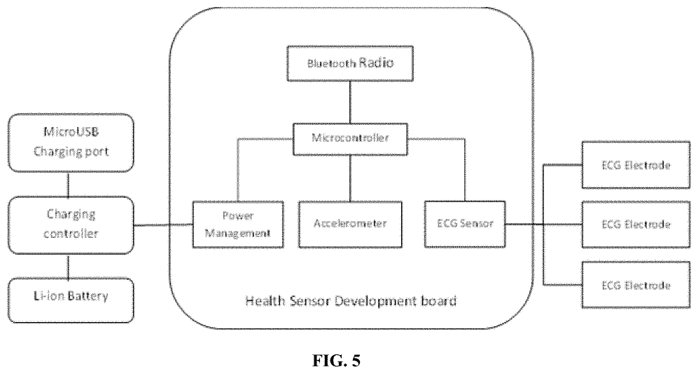

Technical illustrations of the enclosure are shown in FIG. 3 and FIG. 4. The wearable monitoring device may comprise a physical enclosure. The physical enclosure may comprise one or more rigid enclosures. For example, the physical enclosure may comprise two rigid enclosures connected by two hinge joints, which permit the device to contour to the chest of the patient. The two enclosures may house the electronics and a power source of the device (e.g., a rechargeable Li-ion battery). One of the enclosures may comprise a lead with electrode clip, which is configured to provide a reference signal when attached to the chest and allows for noise reduction in the ECG signal. As shown in FIG. 4, the device may comprise a power button 401, ECG clips 405, a sensor board 410, a charging circuit 415, a battery 420, and a charging port 425.

The physical enclosure of the wearable monitoring device may be manufactured using any material suitable for an enclosure, such as a rigid material. The enclosure material may be chosen for one or more characteristics such as bio-compatibility (e.g., non-reactivity, non-irritability, hypoallergenicity, and compatibility with autoclave sterilization), ease of manufacture or processing (e.g., without tooling or other specialized equipment), chemical resistance (e.g., to alkalines, hydrocarbonates, fuels, and solvents), low moisture absorption, mechanical stiffness and rigidity, impact and tensile strength, durability, and low cost. The rigid material may be, for example, a plastic polymer, a metal, a fiber, or a combination thereof. Alternatively, the physical enclosure of the wearable monitoring device may be manufactured using a flexible material, or a combination of a rigid material and a flexible material.

Examples of plastic polymer materials include acrylonitrile butadiene styrene (ABS), polycarbonate (PC), polyphenylene ether (PPE), a blend of polyphenylene ether and polystyrene (PPE+PS), polybutylene terephthalate (PBT), nylon, acetyl, acrylic, Lexan™, polyvinyl chloride (PVC), polycarbonate, polyether, and polyurethane. Examples of metal materials include stainless steel, carbon steel, aluminum, brass, Inconel™, nickel, titanium, and combinations (e.g., alloys or layered structures) thereof. The enclosure may be manufactured or formed by, for example, injection molding or additive manufacturing (e.g., three-dimensional printing). For example, the rigid material may be a rigid, nylon-based material (e.g., DuraForm PA) that can be 3D printed by Selective Laser Sintering (SLS). DuraForm PA may be used due to a number of properties that make it suitable for prototyping medical devices. In particular, the DuraForm PA material may have advantages of ease of manufacture without tooling, good mechanical properties, and suitability for biological purposes.

SLS 3D printing is an additive manufacturing process, which may use a laser to sinter a powdered plastic material based off a three-dimensional (3D) structure. Using SLS 3D printing, custom designs of physical enclosures of the wearable monitoring device may be produced in one-off cycles without a need to produce tooling. Such an approach may allow the device enclosures of the wearable monitoring system to be produced using DuraForm PA at relatively low cost.

The mechanical properties of DuraForm PA may include favorable impact and tensile strengths, which make the material durable. It may be sufficiently rigid enough to protect the electronic components of the device, yet sufficiently flexible enough to prevent cracking when being handled roughly. DuraForm PA also may present good chemical resistance, and may thereby prevent the accidental degradation of the enclosure, such as that caused by exposure to disinfectants or other hospital chemicals.

In addition, DuraForm PA may be tested to be safe for use with humans (e.g., biocompatible) and non-irritating (e.g., to skin where the electrodes are attached). For example, testing performed according to United States Pharmacoepeia (USP) VI standards may demonstrate biocompatibility of this material in vivo.

The physical enclosure of the wearable monitoring device may comprise a maximum dimension of no more than about 5 mm, no more than about 1 cm, no more than about 2 cm, no more than about 3 cm, no more than about 4 cm, no more than about 5 cm, no more than about 6 cm, no more than about 7 cm, no more than about 8 cm, no more than about 9 cm, no more than about 10 cm, no more than about 15 cm, no more than about 20 cm, no more than about 25 cm, or no more than about 30 cm.

For example, the physical enclosure of the wearable monitoring device may comprise a length of no more than about 5 mm, no more than about 1 cm, no more than about 2 cm, no more than about 3 cm, no more than about 4 cm, no more than about 5 cm, no more than about 6 cm, no more than about 7 cm, no more than about 8 cm, no more than about 9 cm, no more than about 10 cm, no more than about 15 cm, no more than about 20 cm, no more than about 25 cm, or no more than about 30 cm.

For example, the physical enclosure of the wearable monitoring device may comprise a width of no more than about 5 mm, no more than about 1 cm, no more than about 2 cm, no more than about 3 cm, no more than about 4 cm, no more than about 5 cm, no more than about 6 cm, no more than about 7 cm, no more than about 8 cm, no more than about 9 cm, no more than about 10 cm, no more than about 15 cm, no more than about 20 cm, no more than about 25 cm, or no more than about 30 cm.

For example, the physical enclosure of the wearable monitoring device may comprise a height of no more than about 5 mm, no more than about 1 cm, no more than about 2 cm, no more than about 3 cm, no more than about 4 cm, no more than about 5 cm, no more than about 6 cm, no more than about 7 cm, no more than about 8 cm, no more than about 9 cm, no more than about 10 cm, no more than about 15 cm, no more than about 20 cm, no more than about 25 cm, or no more than about 30 cm.

The physical enclosure of the wearable monitoring device may have a maximum weight of no more than about no more than about 300 grams (g), no more than about 250 g, no more than about 200 g, no more than about 150 g, no more than about 100 g, no more than about 90 g, no more than about 80 g, no more than about 70 g, no more than about 60 g, no more than about 50 g, no more than about 40 g, no more than about 30 g, no more than about 20 g, no more than about 10 g, or no more than about 5 g.

Adhesives may be used to assemble the wearable monitoring device, such as adhesives supplied by Loctite (Dusseldorf, Germany). Such adhesives may be chosen for characteristics such as suitability for bonding plastics, ability to be cured at room temperature, and certification for biocompatibility and safety for use with humans. These adhesives may be compliant with International Organization for Standardization (ISO) 10993-1 (Biocompatibility Testing).

Electrodes may be used to assemble the wearable monitoring device, such as Red Dot monitoring electrodes with foam tape and sticky gel supplied by the 3M Company (Maplewood, Minn.), or similar electrodes provided by suppliers such as Bio ProTech (Chino, Calif.), Burdick (Mortara Instrument, Milwaukee, Wis.), Covidien (Medtronic, Minneapolis, Minn.), Mortara (Milwaukee, Wis.), Schiller (Doral, Fla.), Vectracor (Totowa, N.J.), Vermed (Buffalo, N.Y.), and Welch Allyn (Skaneateles Falls, N.Y.). Such electrodes may be chosen for characteristics such as suitability for adult patients, with no skin preparation required beforehand, and ability to be clinically tested for several days (e.g., up to 5 days) of usage. In addition, the electrodes may be chosen to have low impedance with ideal electrical properties for the analog-to-digital signal conversion (ADC) performed on the wearable monitoring device.

FIG. 5 shows an example of an electronic system diagram of the wearable monitoring device. The wearable monitoring device may comprise electronic components (electronics) such as a Health Sensor Development board, a charging circuit 415 (e.g., a battery-charging controlling circuit), and a power source or battery 420 (e.g., a rechargeable Li-ion battery). The Health Sensor Development board may comprise components (e.g., sensors and controllers) including a power management integrated circuit (IC), an accelerometer, an onboard ECG sensor, a microcontroller, and a Bluetooth radio circuit. The onboard ECG sensor may be connected via a sensitive amplifier to the three ECG cables to which the ECG electrodes are connected (e.g., via ECG clips 405). The onboard ECG sensor may comprise one or more, two or more, three or more, four or more, five or more, six or more, seven or more, eight or more, nine or more, or ten or more ECG electrodes. The onboard ECG sensor may comprise no more than two, no more than three, no more than four, no more than five, no more than six, no more than seven, no more than eight, no more than nine, or no more than ten ECG electrodes. The power management integrated circuit may be connected to the charging circuit 415 (e.g., charging controller) via an external wire. The external wire may then connect to the Li-ion battery 420 and a charging port 425 (e.g., a MicroUSB charging port). The microcontroller may be connected to, and interface with (e.g., by sending control signals and/or data to, or receiving signals and/or data from), the power management integrated circuit, the accelerometer, the ECG sensor, and the Bluetooth radio integrated circuit.

The monitoring system may provide an end-to-end system for performing (i) capture or recording of measurements of electrical potential at the patient's skin using the ECG electrodes, (ii) conversion of the analog electrical signal into a digital signal within the ECG sensor, (iii) and transmission of data including the digital signal via the Bluetooth radio (e.g., Bluetooth 4.1) and/or antenna.

The Health Sensor Development board of the wearable monitoring device may comprise an off-the-shelf component (e.g., supplied by Maxim Integrated, San Jose, Calif.), which contains a microcontroller unit, a plurality of sensors including the ECG sensor and the accelerometer, a Bluetooth radio, an antenna, and the power management circuitry.

The onboard ECG sensor of the wearable monitoring device may comprise an off-the-shelf component (e.g., a MAX30003 supplied by Maxim Integrated, San Jose, Calif.). The onboard ECG sensor may be an ultra-low power, single channel integrated bio-potential analog front end (AFE) with HR Detection Algorithm (R-R). The onboard ECG sensor may comprise three analog inputs, which correspond to the three input ECG electrodes. The onboard ECG sensor may be configured to have suitable AFE characteristics, such as a suitable clinical grade signal quality, the addition of R-to-R interval and lead-on detection, and low power requirements.

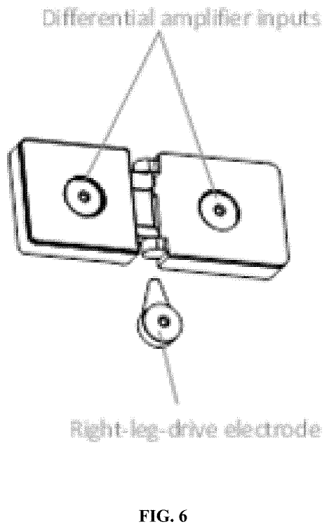

As shown in FIG. 6, the three ECG electrode cables of the wearable monitoring device may correspond to two inputs into a differential amplifier and a reference right-leg-drive electrode configured to provide noise cancellation. The differential amplifier may sense small differences in electrical potential.

To ensure reliability of the wearable electronic device in the event that it is exposed to electrostatic discharge (ESD), the onboard ECG sensor may have electrostatic discharge (ESD) protection. Additionally, the onboard ECG sensor may comprise a low shutdown current to allow for longer battery life.

The onboard ECG sensor of the wearable monitoring device may utilize a high-resolution delta-sigma (ΣΔ) analog to digital converter (ADC) with 15.5 bits of effective resolution, electromagnetic interference filtering (EMI), and a high input impedance (e.g., greater than about 500 MΩ) to maximize signal-to-noise ratio and to ensure a clean ECG signal. The high-resolution ΣΔ ADC may comprise an effective resolution of about 10 bits, about 12 bits, about 14 bits, about 16 bits, about 18 bits, about 20 bits, about 22 bits, about 24 bits, about 26 bits, about 28 bits, about 30 bits, about 32 bits, or more than about 32 bits. The input impedance may be greater than about 50 MΩ, about 100 MΩ, about 200 MΩ, about 300 MΩ, about 400 MΩ, about 500 MΩ, about 600 MΩ, about 700 MΩ, about 800 MΩ, about 900 MΩ, or about 1000 MΩ.

The ECG electrodes of the wearable monitoring device may be a sole point of electronic contact with a patient's body. The points of contact between the patient and the wearable monitoring device may include the ECG electrodes and a temperature sensor. The temperature sensor may be reversibly attached to a surface of the patient's skin to maximize heat transfer between the skin and the sensor. The temperature sensor may be mounted on a retractable, spring-loaded mechanism which protrudes from the patch and presses the sensor to the skin, thereby ensuring a continuous contact between the temperature sensor and the skin in the event of movement. The temperature sensor may also be mounted on a lever constructed from a rigid, yet bendable material to achieve a similar effect. The temperature sensor may be coated with a thermo-conductive material, such as a silicon-based adhesive, to improve heat transfer between the sensor and the skin. The onboard ECG sensor may have a typical leakage current of about 0.1 nanoampere (nA), which is below the patient leakage currents specified in the IEC (International Electrotechnical Commission) 60601-1 standard of 0.1 milliamperes (mA) in normal conditions. The onboard ECG sensor may have a typical leakage current of about 0.01 nA, about 0.05 nA, about 0.1 nA, about 0.5 nA, about 1 nA, about 5 nA, about 10 nA, about 50 nA, about 0.1 microamperes (μA), about 0.5 μA, about 1 μA, about 5 μA, about 10 μA, about 50 μA, or about 0.1 mA.

The accelerometer of the wearable monitoring device may comprise an off-the-shelf component (e.g., an LIS2DH accelerometer supplied by STMicroelectronics, Geneva, Switzerland). The accelerometer may be a microelectromechanical system (MEMS) device offering ultra-low power (e.g., no more than 1 μA, no more than 2 μA, or no more than 4 μA, or no more than 6 μA) and high performance accelerometry data measurement. The accelerometer may be a three-axis linear accelerometer. The accelerometer may allow for the detection of patient activity and movement, informing movement-reduction algorithms applied to the ECG signals captured by the onboard ECG sensor.

Wireless communications of the device may be handled by a wireless transceiver of the wearable monitoring device, which may use off-the-shelf components (e.g., an EM9301 integrated circuit supplied by EM Microelectronic, Colorado Springs, Colo.). The Bluetooth integrated circuit may comprise a fully integrated single-chip Bluetooth Low Energy controller designed for low-power applications (e.g., drawing currents of no more than about 5 mA, no more than about 10 mA, or no more than about 15 mA). The Bluetooth integrated circuit may operate with version 4.1 of the Bluetooth Low Energy protocol, and may controlled by the microcontroller using a standard Bluetooth host controller interface (HCI).

The wearable monitoring device may be powered by a power source, such as an energy storage device. The energy storage device may be or include a solid state battery or capacitor. The energy storage device may comprise one or more batteries of type alkaline, nickel metal hydride (NiMH) such as nickel cadmium (Ni—Cd), lithium ion (Li-ion), or lithium polymer (LiPo). For example, the energy storage device may comprise one or more batteries of type AA, AAA, C, D, 9V, or a coin cell battery. The battery may comprise one or more rechargeable batteries or non-rechargeable batteries. For example, the battery may be a rechargeable, lithium polymer (LiPo) battery. LiPo batteries may be a preferred battery chemistry of choice in many mobile consumer devices, including cell phones. LiPo batteries may provide high energy densities relative to their respective masses; however may include a risk of overheating if appropriate charging methods are not applied. The battery may be, for example, a 3.7 V LiPo battery with 110 milliampere-hours (mAh) of capacity and built-in protection circuitry (e.g., over-charge protection, over-discharge protection, over-current protection, short-circuit protection, and over-temperature protection). The battery may be, for example, a LiPo battery with about 100 mAh, about 200 mAh, about 300 mAh, about 400 mAh, about 500 mAh, about 1000 mAh, about 2000 mAh, or about 3000 mAh of capacity.

The battery may comprise a wattage of no more than about 10 watts (W), no more about 5 W, no more about 4 W, no more about 3 W, no more about 2 W, no more about 1 W, no more about 500 milliwatts (mW), no more about 100 mW, no more about 50 mW, no more about 10 mW, no more about 5 mW, or no more about 1 mW.

The battery may comprise a voltage of no more than about 9 volts (V), no more than about 6 V, no more than about 4.5 V, no more than about 3.7 V, no more than about 3 V, no more than about 1.5 V, no more than about 1.2 V, or no more than about 1 V.

The battery may comprise a capacity of no more than about 50 milliampere hours (mAh), no more than about 100 mAh, no more than about 150 mAh, no more than about 200 mAh, no more than about 250 mAh, no more than about 300 mAh, no more than about 400 mAh, no more than about 500 mAh, no more than about 1,000 mAh, no more than about 2,000 mAh, no more than about 3,000 mAh, no more than about 4,000 mAh, no more than about 5,000 mAh, no more than about 6,000 mAh, no more than about 7,000 mAh, no more than about 8,000 mAh, no more than about 9,000 mAh, or no more than about 10,000 mAh.

The battery may be configured to be rechargeable with a charging time of about 10 minutes, about 20 minutes, about 30 minutes, about 60 minutes, about 90 minutes, about 2 hours, about 3 hours, about 4 hours, about 5 hours, about 6 hours, about 8 hours, about 10 hours, about 12 hours, about 14 hours, about 16 hours, about 18 hours, about 20 hours, about 22 hours, or about 24 hours.

The electronic device may be configured to allow the battery to be replaceable. Alternatively, the electronic device may be configured with a battery which is not replaceable by a user.

In addition, charging current to the battery may be controlled by the charging circuit, which may be configured to monitor battery voltage and to adjust charging currents appropriately.

The mobile application of the monitoring system may provide functionality for a user of the monitoring system to control the monitoring system and a graphical user interface (GUI) for the user to view their measured, collected, or recorded clinical health data (e.g., vital sign data). The application may be configured to run on popular mobile platforms, such as iOS and Android. The application may be run on a variety of mobile devices, such as mobile phones (e.g., Apple iPhone or Android phone), tablet computers (e.g., Apple iPad, Android tablet, or Windows 10 tablet), smart watches (e.g., Apple Watch or Android smart watch), and portable media players (e.g., Apple iPod Touch).

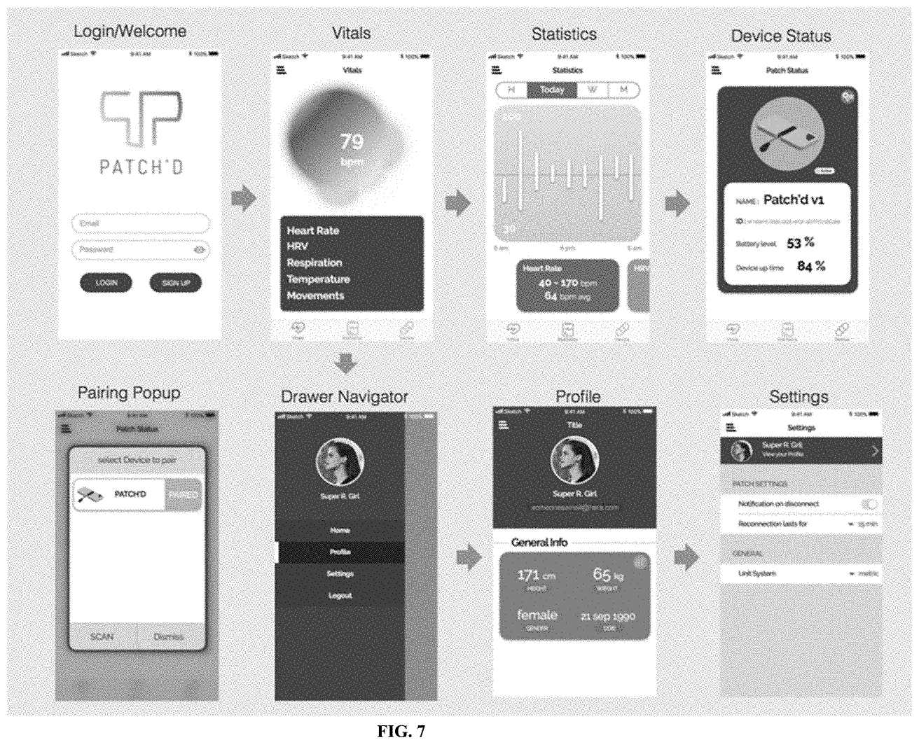

Example mockups of the application graphical user interface (GUI) of the monitoring system are shown in FIG. 7. The application GUI may comprise one or more screens, presenting users with a method of pairing to their wearable monitoring device, viewing (e.g., in real time) their live clinical health data (e.g., vital sign data), and viewing their own trial profile.

The mobile application of the monitoring system may receive data sent from the wearable monitoring device at regular intervals, decode the sent information, and then store the clinical health data (e.g., vital sign data) in a local database on the mobile device itself. For example, the regular intervals may be about 1 second, about 5 seconds, about 10 seconds, about 15 seconds, about 20 seconds, about 30 seconds, about 1 minute, about 2 minutes, about 5 minutes, about 10 minutes, about 20 minutes, about 30 minutes, about 60 minutes, about 90 minutes, about 2 hours, about 3 hours, about 4 hours, about 5 hours, about 6 hours, about 8 hours, about 10 hours, about 12 hours, about 14 hours, about 16 hours, about 18 hours, about 20 hours, about 22 hours, or about 24 hours, thereby provide real-time or near real-time updates of clinical health data. The regular intervals may be adjustable by the user or in response to battery consumption requirements. For example, intervals may be extended in order to decrease battery consumption. The data may be localized without leaving the user's device. The local database may be encrypted, to prevent the exposure of sensitive data (e.g., in the event that the user's phone becomes lost). The local database may require authentication (e.g., by password or biometry) by the user to grant access to the clinical health data and profiles.

Assembly of the wearable monitoring device may comprise a plurality of operations, such as:
1. Soldering of a charging electronic assembly
2. Insertion and attachment of electrode clips into the base of the chassis
3. Connection of two DuraForm PA enclosures at the center hinge
4. Soldering of connecting wires to the charging electronic assembly, health sensor development board, and electrode clips
5. Insertion of the charging circuit electronic assembly, health sensor development board, and the lithium battery into the enclosure
6. Sealing of the enclosure using a biocompatible adhesive
7. Loading of firmware onto the microcontroller
8. System testing

The wearable monitoring device may be designed to provide a functional yet safe hardware with the following features in mind: safety, reliability, accuracy, and usability. The resulting design may be a lightweight, rigid patch with few to no physical hazards. The device may have a total weight of no more than about 1,000 grams (g), no more than about 900 g, no more than about 800 g, no more than about 700 g, no more than about 600 g, no more than about 500 g, no more than about 400 g, no more than about 300 g, no more than about 250 g, no more than about 200 g, no more than about 150 g, no more than about 100 g, no more than about 90 g, no more than about 80 g, no more than about 70 g, no more than about 60 g, no more than about 50 g, no more than about 40 g, no more than about 30 g, no more than about 20 g, no more than about 10 g, or no more than about 5 g.

The device may have no sharp edges or corners, thereby posing little risk of accidental injury or harm (e.g., if dropped or mishandled). The enclosure may be constructed using a rigid material such as DuraForm PA, which is a biocompatible material that may have very low levels of toxicity and irritation. The device may comprise hypoallergenic electrodes, which poses a small risk skin irritation to the user.

The device may be sealed in an enclosure, which is fastened with biocompatible adhesives. Such adhesives may be configured to restrict access to the electronics enclosed inside. The enclosure may act as a barrier to damage of the circuitry and minimize risks of electrical shock or burn from electronic components that may have heated up. The device may comprise a rechargeable lithium ion battery, which may negate the need for a user to perform battery replacement.

The discrete form factor of the patch may allow the patient (user) to perform day-to-day activities with minimum discomfort or interruption, and the strong adhesive provided by the ECG electrodes and the secure ECG clips may prevent the device from becoming disconnected from the user. The device may be safe for children to use because its size, while discrete, may be too large to be swallowed.

Electronic design and component selection of the device may be similarly driven by goals of safety and accuracy. The wearable monitoring device may utilize an off-the-shelf development board (e.g., supplied by Maxim Integrated, San Jose, Calif.), which includes the ECG sensor. Alternatively, the wearable monitoring device may utilize a custom-made printed circuit broad (PCB) including a plurality of components (e.g., supplied by Maxim Integrated, Texas Instruments, Philips, and others).

The device may pose a minute risk of electrocution, since a number of safety features may be included in the health sensor development board and because electrocardiogram is a well-established technology. The ECG sensor forms the electrical connection between the user's body and device via the electrodes. Safety features like defibrillation protection are included, which protects the circuit from being damaged in the event that a patient undergoes defibrillation while wearing the patch, and prevents excessive charge from building up on the device and being discharged into the patient.

Moreover, risk of electric shock may be further reduced by virtue of the wearable monitoring device being battery powered at low voltages (3.7 V). To mitigate the risk to a patient who is wearing the device while charging it, chargers may be provided with short cables that make this practice impractical.

From a radiation perspective, the wearable monitoring device may present very low radiation risk, since it uses Bluetooth Low Energy for wireless communications. Devices using this protocol typically produce radiation emissions measured by Special Absorption Rates (SAR) which are about a thousand times weaker than that of cellphones.

### Computer Systems

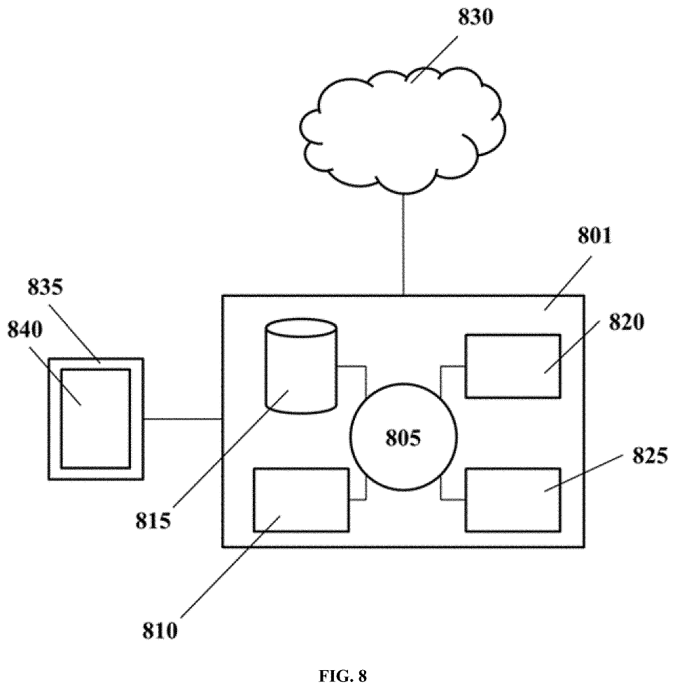

The present disclosure provides computer systems that are programmed to implement methods of the disclosure. FIG. 8 shows a computer system 801 that is programmed or otherwise configured to implement methods provided herein.

The computer system 801 can regulate various aspects of the present disclosure, such as, for example, acquiring health data comprising a plurality of vital sign measurements of a subject over a period of time, storing the acquired health data in a database, receiving health data from one or more sensors (e.g., an ECG sensor) through a wireless transceiver, and processing health data using a trained algorithm to generate an output indicative of a progression or regression of a health condition. The computer system 801 can be an electronic device of a user or a computer system that is remotely located with respect to the electronic device. The electronic device can be a mobile electronic device.

The computer system 801 includes a central processing unit (CPU, also “processor” and “computer processor” herein) 805, which can be a single core or multi core processor, or a plurality of processors for parallel processing. The computer system 801 also includes memory or memory location 810 (e.g., random-access memory, read-only memory, flash memory), electronic storage unit 815 (e.g., hard disk), communication interface 820 (e.g., network adapter) for communicating with one or more other systems, and peripheral devices 825, such as cache, other memory, data storage and/or electronic display adapters. The memory 810, storage unit 815, interface 820 and peripheral devices 825 are in communication with the CPU 805 through a communication bus (solid lines), such as a motherboard. The storage unit 815 can be a data storage unit (or data repository) for storing data. The computer system 801 can be operatively coupled to a computer network (“network”) 830 with the aid of the communication interface 820. The network 830 can be the Internet, an internet and/or extranet, or an intranet and/or extranet that is in communication with the Internet.

The network 830 in some cases is a telecommunication and/or data network. The network 830 can include one or more computer servers, which can enable distributed computing, such as cloud computing. For example, one or more computer servers may enable cloud computing over the network 830 (“the cloud”) to perform various aspects of analysis, calculation, and generation of the present disclosure, such as, for example, acquiring health data comprising a plurality of vital sign measurements of a subject over a period of time, storing the acquired health data in a database, receiving health data from one or more sensors (e.g., an ECG sensor) through a wireless transceiver, and processing health data using a trained algorithm to generate an output indicative of a progression or regression of a health condition. Such cloud computing may be provided by cloud computing platforms such as, for example, Amazon Web Services (AWS), Microsoft Azure, Google Cloud Platform, and IBM cloud. The network 830, in some cases with the aid of the computer system 801, can implement a peer-to-peer network, which may enable devices coupled to the computer system 801 to behave as a client or a server.

The CPU 805 can execute a sequence of machine-readable instructions, which can be embodied in a program or software. The instructions may be stored in a memory location, such as the memory 810. The instructions can be directed to the CPU 805, which can subsequently program or otherwise configure the CPU 805 to implement methods of the present disclosure. Examples of operations performed by the CPU 805 can include fetch, decode, execute, and writeback.

The CPU 805 can be part of a circuit, such as an integrated circuit. One or more other components of the system 801 can be included in the circuit. In some cases, the circuit is an application specific integrated circuit (ASIC).

The storage unit 815 can store files, such as drivers, libraries and saved programs. The storage unit 815 can store user data, e.g., user preferences and user programs. The computer system 801 in some cases can include one or more additional data storage units that are external to the computer system 801, such as located on a remote server that is in communication with the computer system 801 through an intranet or the Internet.

The computer system 801 can communicate with one or more remote computer systems through the network 830. For instance, the computer system 801 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 801 via the network 830.

Methods as described herein can be implemented by way of machine (e.g., computer processor) executable code stored on an electronic storage location of the computer system 801, such as, for example, on the memory 810 or electronic storage unit 815. The machine executable or machine readable code can be provided in the form of software. During use, the code can be executed by the processor 805. In some cases, the code can be retrieved from the storage unit 815 and stored on the memory 810 for ready access by the processor 805. In some situations, the electronic storage unit 815 can be precluded, and machine-executable instructions are stored on memory 810.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 801, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 801 can include or be in communication with an electronic display 835 that comprises a user interface (UI) 840. Examples of user interfaces (UIs) include, without limitation, a graphical user interface (GUI) and web-based user interface. For example, the computer system can include a web-based dashboard (e.g., a GUI) configured to display, for example, patient metrics, recent alerts, and/or prediction of health outcomes, thereby allowing health care providers, such as physicians and treating teams of a patient, to access patient alerts, data (e.g., vital sign data), and/or predictions or assessments generated from such data.

Methods and systems of the present disclosure can be implemented by way of one or more algorithms. An algorithm can be implemented by way of software upon execution by the central processing unit 805. The algorithm can, for example, acquire health data comprising a plurality of vital sign measurements of a subject over a period of time, store the acquired health data in a database, receive health data from one or more sensors (e.g., an ECG sensor) through a wireless transceiver, and process health data using a trained algorithm to generate an output indicative of a progression or regression of a health condition.

### EXAMPLES

### Example 1—Deep Learning Approach to Early Sepsis Detection

A machine learning algorithm is validated for the early prediction of sepsis. The algorithm is capable of operating with a minimal set of easily obtainable vital sign observations and utilizes deep-learning techniques to classify patients.

Dataset

A retrospective analysis is performed on a combined dataset with records from two commonly available research databases: the Multiparameter Intelligent Monitoring in Intensive Care (MIMIC III) database and the eICU Collaborative Research Database. The MIMIC III database is a freely available collection of de-identified patient records from the Beth Israel Deaconess Medical Center between 2001 and 2012. The eICU Collaborative Research Database is a collection of over 200,000 patient records from many critical care facilities located across the U.S. Both databases are made available through PhysioNet, a portal for physiological data made freely available to researchers. Subsets of patients are selected from either database based on the ability to identify the onset of sepsis with a set of selected criteria and to minimize class imbalance problems.

Defining Sepsis Onset

Generally, sepsis refers to an acute non-specific medical condition that lacks a precise method of identification. While it is defined as the dysregulated host response to an infection, in practice, this can be difficult to measure and identify the exact onset of the syndrome. An approach to defining sepsis onset is used according to current Sepsis-3 definitions (e.g., as described by Desautels et al., “Prediction of Sepsis in the Intensive Care Unit With Minimal Electronic Health Record Data: A Machine Learning Approach,” JMIR Med. Informatics, vol. 4, no. 3, p. e28, 2016, which is hereby incorporated by reference in its entirety).

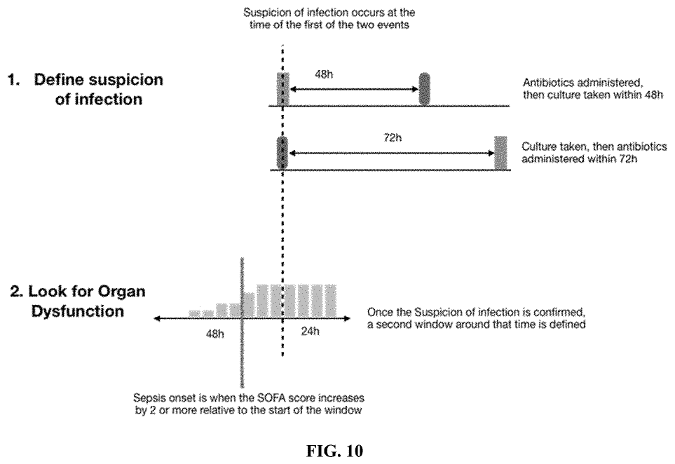

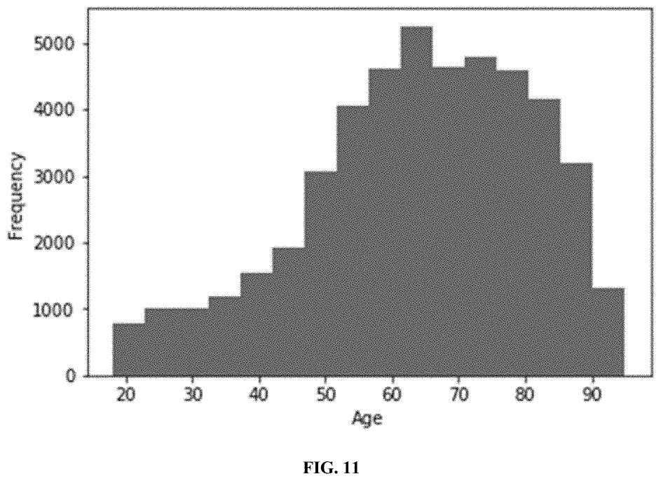

Patients are considered as sepsis-positive if they satisfy the criteria for determining the onset of sepsis. The onset of sepsis is identified as the time when both a suspicion of infection is identified along with an acute change in the SOFA score signifying the dysregulated host response. A suspicion of infection is considered to exist if the combination of lab culture draw and administration of antibiotics occur within a specified time period. If the antibiotics were given first, then the culture must have been drawn within 24 hours. If the culture was drawn first, then the antibiotics must have been given within 72 hours. The time of suspicion is taken as the time of occurrence for the first of the two events. FIG. 10 illustrates an example of defining sepsis onset, such that suspicion of sepsis infection is considered to be present when antibiotics administration and bacterial cultures happen within a defined time period.

To identify an acute change in the SOFA score, a window of up to 48 hours before the suspicion of infection and 24 hours after this time is defined (bounded on either side by the availability of vital sign observations or the end of the stay). The hourly SOFA score is then compared to the value of the SOFA score at the beginning of this window. If the difference in the two scores is at least about 2, then that hour is defined as the onset of sepsis and the patient is considered sepsis-positive.

Exclusion Criteria

Neonates and children are under-represented in the eICU and MIMIC databases; therefore, patients aged under 18 are excluded. Next, hospital admission stays are excluded according to availability of vital signs within a given hospital admission. A stay is excluded if it does not meet the following criteria: (i) at least one observation for heart rate, (ii) at least one observation for respiratory rate, (iii) at least one observation for temperature, and (iv) at least one observation each from two of systolic blood pressure, diastolic blood pressure, blood oxygen concentration (SpO₂).

For patients who are labeled with the ICD-9 code for severe sepsis, an identification of a suspicion of infection and onset time of sepsis were attempted. Patients that are labeled with the ICD-9 code but do not have a suspicion of infection or onset time of sepsis, as calculated from the above method, are excluded.

Due to the varied formats and tendencies of the two databases, database-specific filtering criteria are also applied. In the MIMIC database, data collected from 2001-2008 are excluded by the Carevue due to the underreporting of cultures. Similar to Desautels et al., only data collected by the Metavision system which was used at the Beth Israel Deaconess Medical Center from 2008 onward are selected.

When the eICU patient stays are examined, only 4,758 of the total number of patients satisfy the onset criteria. In order to avoid a significant class imbalance, 18,760 patients who did not meet the onset criteria are selected.

The final cohort includes a total of 47,847 patients. Of these, 13,703 patients (28.6%) are labeled with sepsis and a time-onset. Further, 24,329 (50.8%) of these patient stays are derived from the MIMIC III database and 23,518 (49.2%) are derived from the eICU database (as shown in Table 1). FIG. 11 illustrates an age distribution histogram of a selected cohort.

TABLE 1
Numbers of patients for sepsis patients and non-sepsis
patients derived from the MIMIC III and eICU databases

| | Non-Sepsis | Sepsis | Total |
|---|---|---|---|
| MIMIC III | 15,384 | 8,945 | 24,329 |
| eICU Collaborative | 18,760 | 4,758 | 23,518 |
| Research Database | | | |
| Total | 34,144 | 13,703 | 47,847 |

Machine Learning Using Recurrent Neural Networks

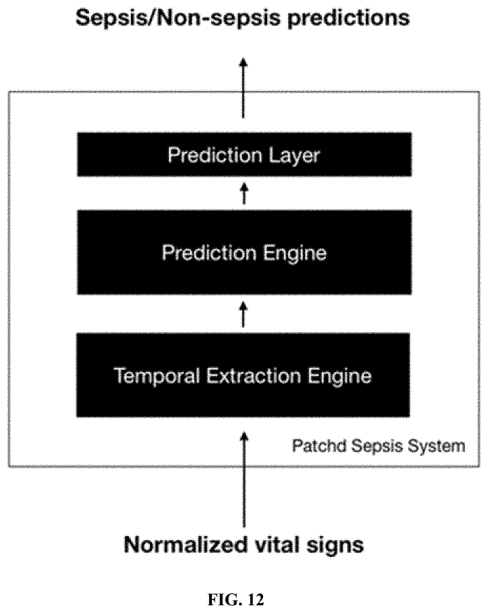

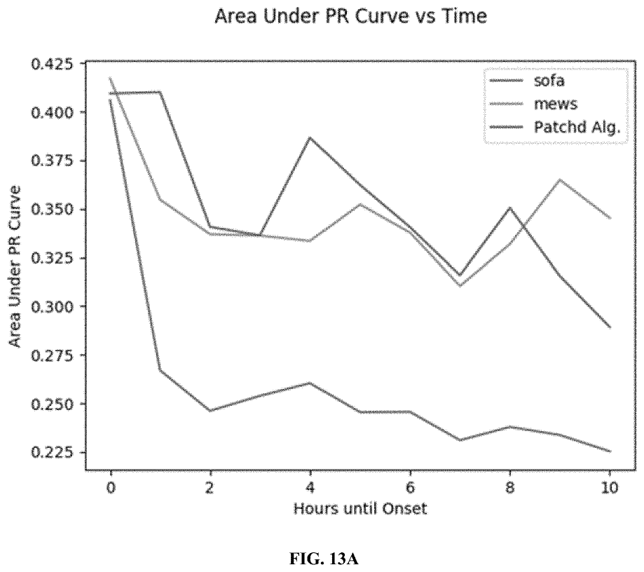

A Machine Learning Algorithm Comprising a Machine-Learning Based Classification Engine is developed, which is capable of predicting the early onset of sepsis. The algorithm architecture is based on an artificial neural network (ANN). As illustrated in FIG. 12, the machine learning algorithm for predicting sepsis from normalized vital signs comprises a temporal extraction engine, a prediction engine, and a prediction layer.

The temporal extraction engine utilizes a recurrent neural network (RNN) to derive temporal based insights from a set of inputs comprising one or more vital signs (e.g., normalized vital signs). The RNN comprises multiple stacked layers long short-term memory (LSTM) units which retain information over arbitrary time intervals.

Algorithm inputs comprise vital sign observations and demographic covariates. Commonly measured vital signs, including heart rate, temperature, diastolic blood pressure, systolic blood pressure, respiratory rate and blood oxygen concentration (SpO₂), are used to generate predictions. Examples of covariate variables include age and sex.

To further minimize class imbalance problems, sepsis-positive cases are augmented to allow for a greater proportion of sepsis-positive to sepsis-negative cases. Within a sepsis-positive stay, vital sign observations occurring at the same time have their order rearranged, and the time of sepsis onset is increased or decreased by a randomly selected interval between −2 hours and +2 hours.

To perform training of the machine learning architecture, the set of patient stays is divided into two sets, from which training samples are selected: sepsis-positive and sepsis-negative. From the sepsis-positive stays, vital sign observations which occur after the onset of sepsis are discarded. Multiple training samples are selected based on the length of the stay.

Training and testing is performed using the Tensorflow deep learning software library on cloud computing GPU-based infrastructure provided by Amazon Web Services.

Validation

The dataset is split into separate training, development, and test sets comprising 34,408, 6,611, and 6,828 patient stays, respectively. Data for each set are selected randomly from the cohort, as illustrated in the set allocation listed in Table 2.

TABLE 2
Distribution of admissions

| Set | No. admissions | Proportion |
|---|---|---|
| Training | 34,408 | 71.9% |
| Development | 6,611 | 13.8% |
| Test | 6,828 | 14.3% |
| Total | 47,847 | 100% |

As sepsis is frequently diagnosed at or shortly after admission into a hospital (e.g., an intensive care unit, ICU), the variable length of data preceding sepsis onset is accounted for using a form of case-control matching. The length of sepsis-negative patient sequences is varied to match those of sepsis-positive patients. Sepsis-positive patients are arranged by hospital admissions in ascending order of time from first vital sign observation to sepsis, and are paired with sepsis-negative patient stays in a ratio of 1 to 4. Sepsis-negative sequences are then sampled from the sepsis-negative stay with a length equaling that of its matched sepsis-positive stay.

After training, the performance of the training algorithm is tested on the development set to determine algorithm performance. The average area under the precision-recall curve (AUPRC) and average area under the receiver operator characteristic curve (AUROC) over the last five hours before sepsis-onset are taken as a two-variable metric, against which the algorithm is optimized.

Final validation is performed on the test set on which a plurality of performance metrics are derived, including sensitivity (recall), specificity, precision (positive predictive value, PPV), true positive rate, false positive rate, true negative rate, and false negative rate. Algorithm performance is then compared to other sepsis-diagnosis tools, the SOFA and MEWS scores.

Algorithm Performance

The machine learning algorithm is trained on the combined dataset generated from the MIMIC III and EICU critical care database. Predictions are then generated for the test set patients. In examining the performance of the algorithm, a first consideration can include how the algorithm performs across all thresholds.

Measures of AUPRC and AUROC provide indicators of algorithm performance summed across many different operating points for the machine learning algorithm. AUPRC provides a focus on the ability of the algorithm to identify true positives and provides insight as there is a class imbalance problem. AUROC is provided to demonstrate algorithm efficacy in the case of true negatives. Both methods aim to provide a measure of overall algorithm performance.

Receiver operating characteristics are generated at the time of sepsis onset and at 2, 4, 6, 8, and 10 hours preceding the onset of sepsis. At sepsis onset, the machine learning algorithm achieves an AUROC of 0.684, and at four hours prior to sepsis onset, the machine learning algorithm achieves an AUROC of 0.663. These values exceed the corresponding AUROC (at sepsis onset and at four hours prior to sepsis onset) for SOFA scores (0.642 and 0.516, respectively) and for MEWS scores (0.653 and 0.590, respectively). At each time before sepsis onset, the area under the curve (AUPRC) is calculated (as illustrated in Table 3). Similar results are derived for the area under the receiver-operating characteristic (AUROC) (as illustrated in Table 4).

TABLE 3
Area Under the Precision Recall Curve (AUPRC) for the
machine learning algorithm at varied hours prior to sepsis
AUPRC

| Hours Prior | Machine Learning | | |
|---|---|---|---|
| to Sepsis | Algorithm | SOFA | MEWS |
| 0 | 0.409 | 0.406 | 0.417 |
| 2 | 0.341 | 0.246 | 0.337 |
| 4 | 0.387 | 0.260 | 0.333 |
| 6 | 0.341 | 0.246 | 0.338 |
| 8 | 0.350 | 0.238 | 0.332 |
| 10 | 0.289 | 0.225 | 0.345 |

TABLE 4
Area Under Receiver Operating Characteristic (AUROC) for the
machine learning algorithm at varied hours prior to sepsis
AUROC

| Hours Prior | Machine Learning | | |
|---|---|---|---|
| to Sepsis | Algorithm | SOFA | MEWS |
| 0 | 0.684 | 0.642 | 0.653 |
| 2 | 0.660 | 0.504 | 0.604 |
| 4 | 0.663 | 0.516 | 0.590 |
| 6 | 0.659 | 0.523 | 0.608 |
| 8 | 0.672 | 0.503 | 0.598 |
| 10 | 0.659 | 0.528 | 0.609 |

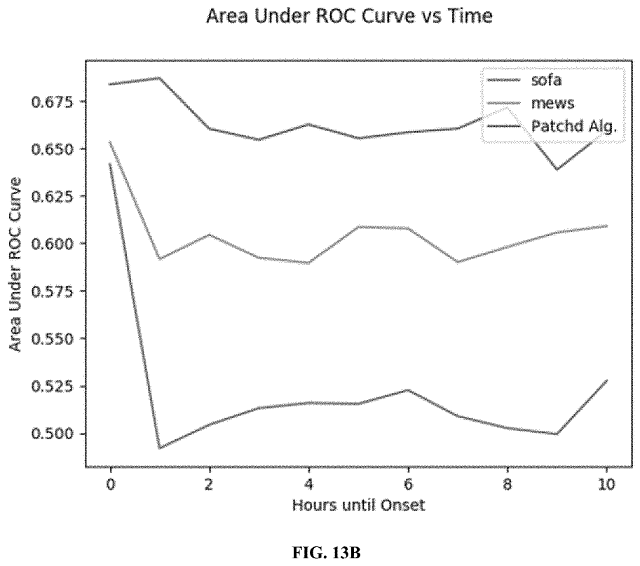

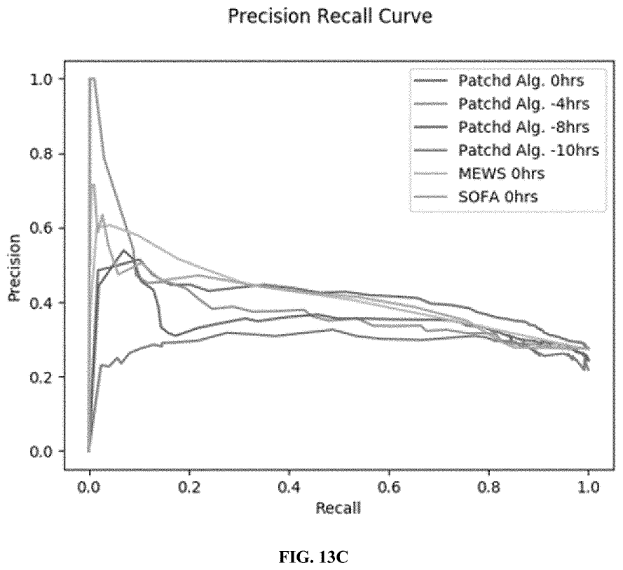

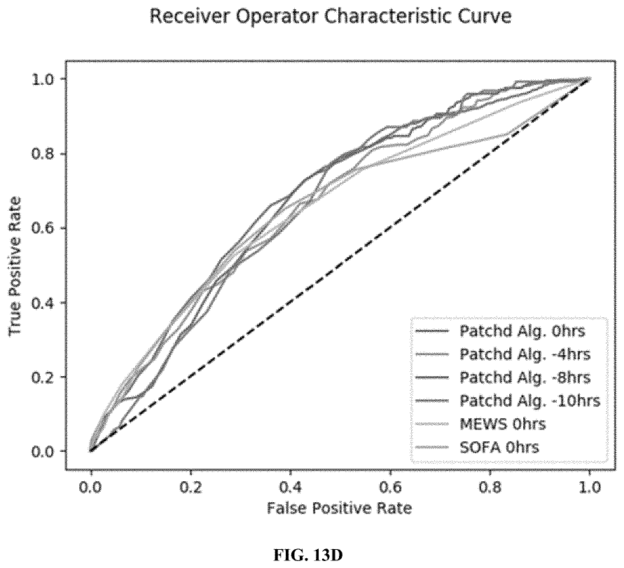

FIG. 13A illustrates an area under the precision-recall (PR) curve vs. time. FIG. 13B illustrates an area under the receiver operator characteristic (ROC) curve vs. time. FIGS. 13C-13D illustrate precision-recall (PR) and receiver operating characteristic (ROC) curves, respectively, plotted at different times for a sepsis prediction algorithm vs. the prediction made by the SOFA and MEWS scores at the onset of sepsis. Note that the sepsis prediction algorithm generates an ROC that is comparable to the existing measures, the SOFA and MEWS scores.

Classification Threshold Selection and “Real World” Performance

While measures of AUPRC and AUROC provide indicators of overall algorithm performance, they may not reflect what predictions may be made in a real-world application. To determine the real-world performance of the algorithm, a classification threshold is selected that maximizes precision and sensitivity. The specific performance metrics are then derived at each time period (as illustrated in Table 5).

TABLE 5
Performance metrics of the machine learning algorithm at
varied hours prior to sepsis

| Hours Prior | | | | | | |
|---|---|---|---|---|---|---|
| to Sepsis | 0 | 2 | 4 | 6 | 8 | 10 |
| True Positive | 765 | 400 | 273 | 196 | 152 | 105 |
| True Negative | 1035 | 744 | 529 | 386 | 330 | 188 |
| False Positive | 1383 | 854 | 590 | 429 | 299 | 253 |
| False Negative | 159 | 108 | 87 | 56 | 49 | 18 |
| Total Patients | 3342 | 2106 | 1479 | 1067 | 830 | 564 |
| Precision | 0.356 | 0.319 | 0.316 | 0.314 | 0.337 | 0.293 |
| Recall/ | 0.828 | 0.787 | 0.758 | 0.778 | 0.756 | 0.854 |
| Sensitivity | | | | | | |
| False Positive | 0.428 | 0.466 | 0.473 | 0.474 | 0.525 | 0.426 |
| Rate | | | | | | |
| Specificity | 0.428 | 0.466 | 0.473 | 0.474 | 0.525 | 0.426 |

Although the description has been described with respect to particular embodiments thereof, these particular embodiments are merely illustrative, and not restrictive. Concepts illustrated in the examples may be applied to other examples and implementations.

### Example 2—A Deep-Learning Model for Early Sepsis Detection with a Minimal Non-Invasive Set of Physiological Inputs

Abstract

Early sepsis intervention may be crucial for patient outcomes and cost reduction. Machine learning and artificial intelligence methods may provide an opportunity for improving the accuracy of early sepsis detection, thereby allowing for quicker time to administer effective treatment, such as resuscitation and antibiotic administration, to patients with sepsis. Results of an observational cohort study demonstrated that a deep-learning model is capable of identifying sepsis up to 8 hours prior with a minimal set of non-invasive vital sign inputs. A retrospective cohort study was conducted using open source intensive care unit (ICU) data. The ICU data included patient stays from two open-source datasets with data sourced from multiple centers from across the US from 2001-2015, and were obtained from predominantly adult patients that were included in the MIMIC-III Critical Care Database and the eICU Collaborative Research Database. The data were randomly divided into a training data set, a development data set, and a validation data set, and these data sets were used to train a deep-learning model.

The deep-learning model was validated on a set of 3,426 intensive care unit (ICU) patients, including positive sepsis cases and negative sepsis cases. The performance of the model in detecting sepsis onset was quantified using two measures, the area under the receiver-operator characteristic curve (AUROC) and the area under the precision-recall curve (AUPRC). The AUROC is a performance metric for assessing binary diagnostic tests and machine-learning classifiers. The AUPRC adds further performance information in light of the low proportion of sepsis-positive patients within the dataset. The deep-learning model achieved an AUROC of 0.84, which exceeded that of existing standard-of-care measures (AUROC of 0.68).

Notably, deep-learning model incorporating systems and methods of the present disclosure outperformed existing risk scores of SOFA, qSOFA, SIRS, and MEWS in detecting sepsis onset, as indicated by AUROC and AUPRC (P<0.001). The deep-learning model algorithm detected sepsis onset with an AUROC of 0.84±0.01 (standard deviation) at sepsis onset, thereby outperforming each of the risk scores (SOFA: AUROC of 0.68; qSOFA: AUROC of 0.57; SIRS: AUROC of 0.62; and MEWS: AUROC of 0.68). Even when performed using data obtained from patients at least 8 hours prior to the onset of sepsis (as defined by Sepsis-3), the deep-learning model algorithm detected sepsis onset with an AUROC of 0.72, which outperformed the existing risk scores performed on data obtained at sepsis onset. Further, the deep-learning model algorithm detected sepsis onset with an AUPRC of 0.67 at sepsis onset, which exceeded that of the risk scores (SOFA: AUPRC of 0.36; qSOFA: AUPRC of 0.28; SIRS: AUPRC of 0.30; and MEWS: AUPRC of 0.39).

These results demonstrated that a deep learning model trained using a set of six vital signs and demographic information can outperform standard-of-care measures, even up to 8 hours prior to sepsis onset.

Introduction

Sepsis is one of the leading causes of mortality in U.S. hospitals. In the U.S., sepsis is estimated to affect 1.7 million Americans annually with mortality rates reaching 270,000. Defining sepsis and best practice for its management may be an evolving topic (ranging from sepsis phenotypes, to immunoprophylaxis, to infection tolerance). As used herein, the term “sepsis” refers to a dysregulated host response to infection.

Despite the large number of sepsis cases annually in the U.S., sepsis risk may vary in specific patient populations. For example, the relative risk in a cancer patient is about four times that of non-cancer patients, and as high as 65 times that of non-cancer patients for patients with myeloid leukemia. While the impact of sepsis is often evaluated through mortality rate in an acute care setting, the impact of sepsis often extends beyond the hospital. Sepsis survivors may experience increased morbidity and have significantly worse long-term outcomes; further, sepsis may create a financial burden for providers and payers, both in the short and long term. Unsurprisingly, this financial burden may correlate with sepsis severity.

In some cases, delayed antibiotic administration to sepsis patients, even by one hour, can lead to significant increases in mortality risk. This finding may lead to a significant focus on managing sepsis in the acute care setting. However, the beginning symptoms of sepsis may be present before admission. In a retrospective chart review across four hospitals in New York, the Centers for Disease Control and Prevention (CDC) found that 79.4% of sepsis cases were present at admission. As a consequence, the need for sepsis detection in a post-acute setting may be key in reducing sepsis mortality.

In an effort to address acute care sepsis, a number of methods may be performed to identify and triage patients. These range from bacterial cultures to risk scores based on commonly observed vital signs and lab measurements. A key example of this is the Sepsis-3 scoring system. In 2017, a sepsis task force was established to evaluate methods for identifying sepsis. The group established the use of the sequential organ failure assessment (SOFA) and its simplified counterpart, the quick sequential organ failure assessment (qSOFA), in patients with suspected infection, as an ideal measure for identifying patients with sepsis. The SOFA and qSOFA scores quantify the severity of organ failure. Other risk scores include the modified early warning score (MEWS), Acute Physiology and Chronic Health Evaluation Score (APACHE score) and the Simplified Acute Physiology Score (SAPS), which focus more broadly on mortality risk. Despite the use of these scores, the early detection of sepsis may remain a challenging problem in medicine, especially in the post-acute care setting. Methods and systems of the present disclosure may incorporate machine learning techniques such as deep-learning models to achieve early detection of sepsis with excellent performance, as indicated by metrics such as AUROC and AUPRC.

Machine learning methods may be applied toward diagnosis or prediction of sepsis. For example, neural networks may be used to predict septic shock. Threshold-based heuristics (e.g., with Bayesian inference) may be used to detect sepsis onset up to 3 hours prior to the onset. The same group further advanced their approach by using Bayesian inference. Other techniques may use various machine learning techniques, such as logistic regression, support vector machines, deep learning, neural networks, gradient boosting, dynamic Bayesian networks, K-nearest neighbors, naive Bayes, and logistic regression with L2 regularization. For example, Gaussian processes may be combined with recurrent neural networks (RNNs) to predict sepsis, based on inputs such as lab results, drug administration, patient demographics, and vital sign observations. Sepsis may be investigated and evaluated using data obtained from patients in ICU and Emergency Department (ED) settings, including input variables such as vital signs, laboratory tests, drug administration, nursing chart assessments, and other clinical events.

A retrospective study was performed to validate a deep-learning approach for the early prediction of sepsis. The Deep Learning Algorithm (DLA) may be used to predict the onset of sepsis more accurately than existing standards of care using a minimal set of vital sign observations which do not require lab tests and can easily be obtained in a post-acute setting. The algorithm was validated for data obtained from patients up to 8 hours prior to onset as defined by Sepsis-3, and performance of the sepsis detection was assessed using metrics such as the area under the receiver operating characteristic curve (AUROC). In addition, performance was evaluated with area under the precision-recall curve (AUPRC), a metric which provides important information regarding the algorithm's ability to classify when there are comparatively fewer positive samples in the population.

Dataset

A retrospective analysis was performed on a combined dataset with records from two commonly available research databases made available through PhysioNet32. The databases included the Multiparameter Intelligent Monitoring in Intensive Care (MIMIC III) database and eICU Collaborative Research Database. The MIMIC III database is a freely available collection of de-identified patient records from the Beth Israel Deaconess Medical Center between 2001 and 2012. The eICU Collaborative Research Database is a collection of over 200,000 patient records from many critical care facilities located across the U.S.

Defining Sepsis Onset

Sepsis is an acute non-specific medical condition that lacks a precise method for detecting the exact onset of the syndrome. For this study, the current Sepsis-3 definitions were used, which may be an improvement over a previous definition, Sepsis-235.

Patients were considered as sepsis-positive if they satisfied the Sepsis-3 criteria for sepsis. Sepsis onset was then identified as the time when there is both suspicion of infection and an acute change in the SOFA score, signifying the dysregulated host response. Suspicion of infection was considered to exist if a lab culture draw and administration of antibiotics occurred within a specified time period. If antibiotics were given first and a culture was drawn within 24 hours, then a suspicion of infection was considered to be present. Alternatively, if the culture was drawn first and the antibiotics were administered within 72 hours, then that was also considered a suspicion of infection. The time of suspicion was taken as the time of occurrence for the first of the two events (as shown in FIG. 10).

To identify an acute change in the SOFA score, a time window was defined from 48 hours before the suspicion of infection to 24 hours after the suspicion of infection (bounded on either side by the availability of vital signs). The hourly SOFA score was then compared to the value of the SOFA score at the beginning of this window. If there was an increase in the score of more than two, then that hour was defined as the onset of sepsis and the patient was considered sepsis-positive.

Exclusion Criteria

Pediatrics are under-represented in the eICU and MIMIC databases; therefore, the majority of the population is adult. Since sepsis presents differently between adults and children, this study demonstrates how the algorithm performs in adult patients. Hospital admission stays were excluded according to the availability of vital signs within a given hospital admission. A stay was excluded if it did not meet the following criteria: at least one observation for heart rate, at least one observation for respiratory rate, at least one observation for body temperature, and at least one observation for at least two of systolic blood pressure, diastolic blood pressure, and blood oxygen concentration (SpO₂).

Patients who were labeled with the ICD code for severe sepsis but did not meet either the suspicion of infection or acute change in SOFA score criteria were excluded from the study.

Due to the varied formats and tendencies of the two databases, database-specific filtering criteria were also applied. The data collected from 2001-2008 by the CareVue system were excluded due to the underreporting of cultures.

When the eICU patient stays were examined, only 4,758 of the total number of patients satisfied the Sepsis-3 criteria. To avoid a significant class imbalance between sepsis-positive and sepsis-negative patients, a subset of 23,518 patients who did not meet the Sepsis-3 criteria were chosen such that roughly equal proportions would be chosen between the eICU and MIMIC III databases.

The final cohort included a set of 47,847 patients. Of these, 13,703 patients (28.6%) were labeled with sepsis and a time-onset of sepsis. Of the total dataset, 24,329 patient stays (50.85%) were derived from the MIMIC III database and 23,518 patient stays (49.15%) were derived from the eICU database. A full list of patient demographics are listed in Table 6.

TABLE 6
Demographics of final cohort of patients

| | | Count | Percentage |
|---|---|---|---|
| ICU source | EICU | 23,518 | 49.15 |
| | MIMIC-III | 24,329 | 50.85 |
| Gender | Male | 26,422 | 55.22 |
| | Female | 21,417 | 44.76 |
| | Unknown | 8 | 0.02 |
| Age | <18 | 102 | 0.21 |
| | 18 ≤ x < 30 | 2,215 | 4.63 |
| | 30 ≤ x < 40 | 2,503 | 5.23 |
| | 40 ≤ x < 50 | 4,488 | 9.38 |
| | 50 ≤ x < 60 | 8,525 | 17.82 |
| | 60 ≤ x < 70 | 10,767 | 22.50 |
| | 70 ≤ x | 19,246 | 40.22 |
| | Unknown | 1 | 0.01 |
| Ethnicity | Unknown/Other | 3,688 | 7.71 |
| | Asian | 1,007 | 2.10 |
| | White | 36,093 | 75.43 |
| | Hispanic | 1,746 | 3.65 |
| | African American | 5,113 | 10.69 |
| | Native American | 193 | 0.40 |
| | Pacific Islander | 7 | 0.01 |
| Death | Yes | 4,698 | 9.82 |
| | No | 43,149 | 90.18 |

Deep-Learning Model

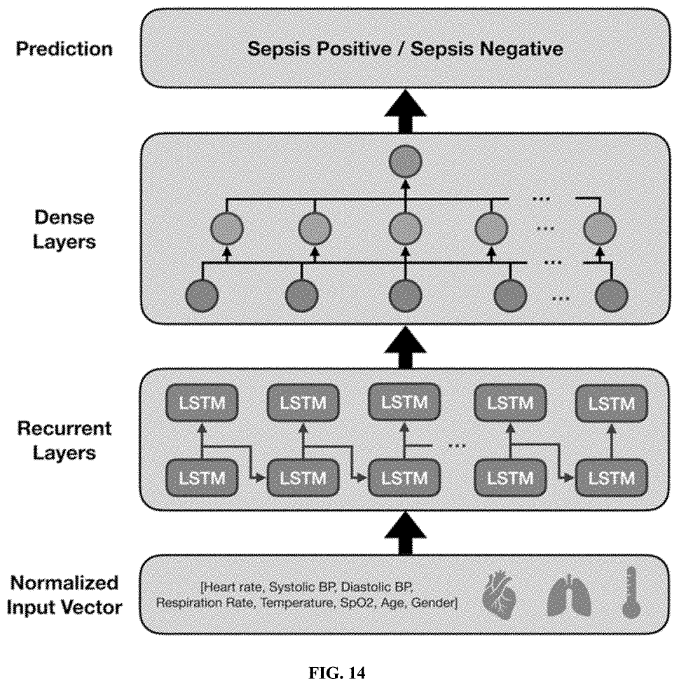

The deep learning algorithm (DLA) is a machine learning based classification engine capable of predicting the early onset of sepsis. The deep learning algorithm comprises four major components, as shown in FIG. 14. The first component comprises the input component, where vital signs and demographic information are normalized and fed into the models as an input vector. The second component comprises a recurrent neural network (RNN) layer to model the time-dependent relationships within the data, in which stacked LSTM layers are used. The third component comprises a set of dense layers, where the representations of the data from the recurrent neural networks are combined together. The number of hidden units and layers may be tuned as hyper-parameters. The fourth component comprises a prediction layer, which determines a prediction indicative of whether a patient is sepsis-positive or sepsis-negative.

The deep learning algorithm was trained using training data set, where the inputs comprised a set of vital sign observations and demographic covariates. The set of vital signs observations used to generate predictions included those that are commonly measured in clinical settings: heart rate, temperature, diastolic blood pressure, systolic blood pressure, respiratory rate, and blood oxygen concentration (SpO₂). Covariate variables included the age and gender of each patient.

The recurrent neural network (RNN) was used to derive temporal features from the set of vital sign inputs. The RNN comprised a plurality of stacked layers of long short-term memory (LSTM) units which retain information over arbitrary time intervals.

Training and testing were performed using the Tensorflow deep learning software library on a cloud computing GPU-based infrastructure provided by Amazon Web Services.

Data Processing

The dataset was split into separate training data sets, development data sets, and test data sets comprising 80%, 10%, and 10% of all patient stays, respectively. The training data set was used to train the deep learning model, and the development data set was used to estimate the model's performance and to compare performance across different models. The test data set was set aside for final evaluation.

The training data set was divided into two subsets: a first subset comprising sepsis-positive patient stays and a second subset comprising sepsis-negative patient stays. Among the first subset comprising sepsis-positive patient stays, vital sign observations which occurred after the onset of sepsis were discarded.

To increase the amount of training data, patient data points were augmented by removing 10% of measurements or by removing all measurements of one type. Additionally, the time of sepsis onset was randomly adjusted between two hours before and two hours after the defined onset for the sepsis-positive patients. To minimize class imbalance in the training set, the batches for the positive patients were chosen so that they would overlap, allowing more batches to be selected. From the second subset comprising sepsis-negative patient stays, multiple training samples were selected randomly from each stay. The deep learning model was trained end-to-end using backpropagation.

After training was completed, the trained deep learning model was tested on the development data set to assess the performance of the deep learning algorithm. This process was repeated for different sets of model hyperparameters, including network size and learning rate. Because of the large state-space of hyperparameters, this process was repeated and tuned using a variation of the optometrist algorithm, a method which uses a combination of stochastic processes and human decision to select hyperparameters. Next, the set of hyperparameters that produced the greatest performance of the deep learning algorithm was selected for the final model, which was then validated on the test data set.

Processing of the test data set required further matching and filtering. As sepsis is frequently diagnosed for a given patient at or shortly after the patient's admission into the ICU, the amount of available data prior to onset of sepsis can be limited among sepsis-positive patients. This limitation does not exist in sepsis-negative patients, so this was accounted for by matching the length of sepsis-negative patient sequences with those of sepsis-positive patients. The set of sepsis-positive hospital stays was arranged in descending order of length of stay, and then paired with the set of sepsis-negative patient stays, at a ratio of approximately 1 to 4. For each negative-positive patient pairing, sepsis-negative sequences were randomly sampled from the stay with a length equal to the matched sepsis-positive stay. For example, if a given sepsis-positive patient among a negative-positive patient pairing developed sepsis after being in the ICU for 5 hours, only 5 hours worth of data was taken at random from the corresponding negative-sepsis patient among the negative-positive patient pairing. Patients were omitted from the study based on certain criteria, such as if the hospital stay was too short or if the density of vital sign observations was either too high or too low.

Validation

Final evaluation was performed on the test data set by performing a comparison of the performance of the deep learning algorithm with that of current standard-of-care scores: SOFA, qSOFA, SIRS, and MEWS.

To estimate the variance of the performance characteristics, the training data sets and the test data sets were resampled using the bootstrapping method. Performance metrics, including the area under the precision-recall curve (AUPRC) and the area under the receiver operator characteristic (AUROC) were calculated to assess the performance of the deep learning algorithm. The AUPRC provides a performance characteristic for the ability of the algorithm to identify true positives, and is relevant in this study because the large number of sepsis-negative patients relative to sepsis-positive patients presents a class imbalance.

Precision-recall curves and receiver operating characteristics were evaluated at sepsis onset and at multiple time-points preceding it. To generate the ROC at 2 hours before sepsis onset, only data up to and including that time point were used. This was repeated for 4, 6, and 8 hours before sepsis onset. From these curves, the AUROC and AUPRC were calculated for the deep learning algorithm and for the set of four standard-of-care measures, SOFA, qSOFA, SIRS, and MEWS.

The AUPRC and AUROC provided indicators of overall algorithm performance across many operating thresholds. Application in a clinical setting requires that an operating threshold be selected, which means the algorithm can be tuned to minimize false positives, for example. In this study, the algorithm performance was examined at two potential classification thresholds (0.5 and 0.9) selected to illustrate the ability to tune the sensitivity, specificity, and positive predictive value (PPV) of the deep learning algorithm. For example, with a classification threshold of 0.5, all prediction values greater to or equal to 0.5 are classified as a sepsis-positive outcome, while all prediction values less than 0.5 are classified as a sepsis-negative outcome. As another example, with a classification threshold of 0.9, all prediction values greater to or equal to 0.9 are classified as a sepsis-positive outcome, while all prediction values less than 0.9 are classified as a sepsis-negative outcome. As the classification threshold increases, the sensitivity of the classification is expected to decrease, while the specificity and the positive predictive value of the classification are expected to increase. Similarly, as the classification threshold decreases, the sensitivity of the classification is expected to increase, while the specificity and the positive predictive value of the classification is expected to decrease.

Results

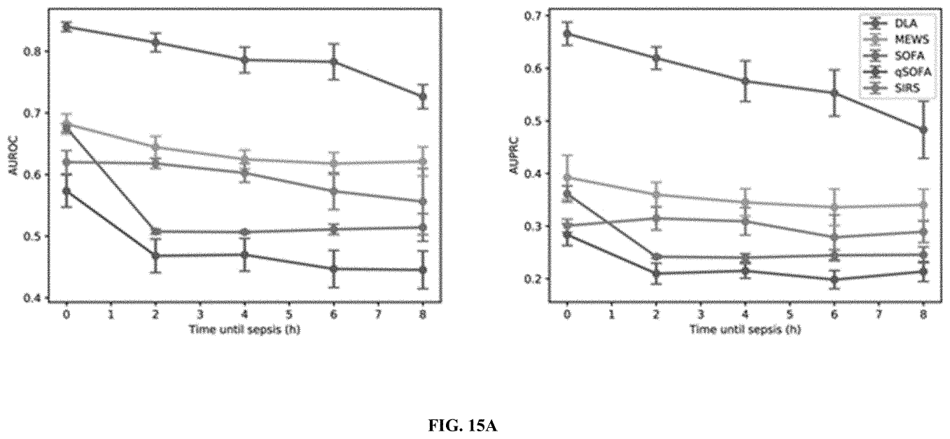

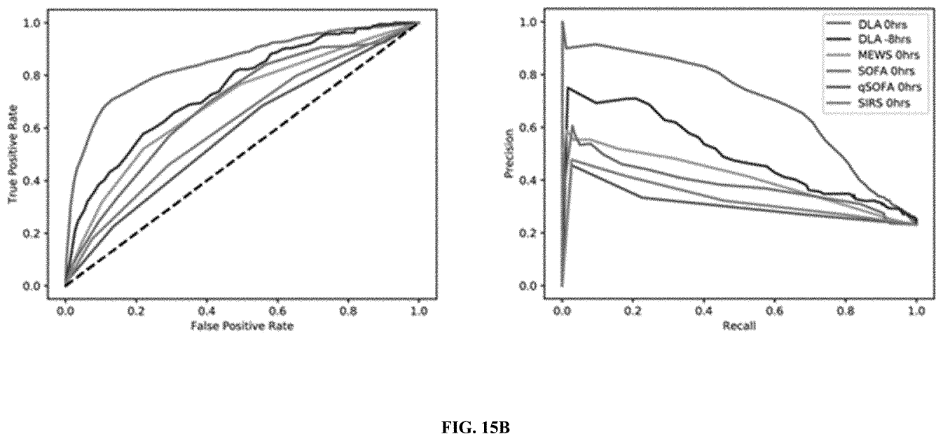

FIGS. 15A-15B illustrate a comparison in performance between the deep learning algorithm (DLA) and a set of four risk score approaches to predicting sepsis onset (MEWS, SOFA, qSOFA (quick SOFA), and SIRS (Systemic Inflammatory Response Syndrome)). FIG. 15A illustrates plots of AUROC vs. time (left) and AUPRC vs. time (right) for the DLA and four risk score approaches (MEWS, SIRS, SOFA, and qSOFA). The x-axis indicates time prior to the onset on a 1-hour scale, the y-axis shows the performance of each metric, and each of the plotted curves represents the performance metric for the different approaches for predicting sepsis onset (from top to bottom: DLA, MEWS, SIRS, SOFA, and qSOFA). FIG. 15B illustrates a receiver-operator characteristic (ROC) curve (left) and a precision-recall curve (PRC) (right) for the DLA plotted at sepsis onset and at 8 hours before, as well as the comparison to the four risk score approaches to predicting sepsis onset (MEWS, SOFA, qSOFA, and SIRS) onset. The x-axis shows the false positive rate for the ROC (left) or the recall for the PRC (right), the y-axis shows the true positive rate for the ROC (left) or the precision for the PRC (right), and each of the plotted curves represents the ROC (left) or PRC (right) for the different approaches for predicting sepsis onset (from top to bottom: DLA at sepsis onset (0 hours prior), DLA at 8 hours prior to sepsis onset, MEWS at sepsis onset, SOFA at sepsis onset, SIRS at sepsis onset, and qSOFA at sepsis onset).

The deep learning algorithm achieved an AUROC of 0.84±0.01 (standard deviation) and an AUPRC of 0.67±0.02 (standard deviation) for predicting sepsis onset at the current time. The deep learning algorithm outperformed each of the four scoring-based assessments for sepsis onset based on both the AUPRC (p<0.001) and the AUROC (p<0.001) measures of performance (as shown in FIGS. 15A and 15B).

While the performance of the deep learning algorithm decreased as the time of prediction moved away from the onset of sepsis, at 8 hours before onset, the deep learning algorithm achieved an AUROC of 0.73±0.02 and an AUPRC of 0.48±0.05 which exceeded the performance of each risk score-based assessment at sepsis onset (SOFA: AUROC of 0.68; qSOFA: AUROC of 0.57; SIRS: AUROC 0.62; and MEWS: AUROC of 0.68). This difference may have a significant impact toward achieving sepsis prediction that has high clinical utility and is clinically actionable, given that a random classifier (e.g., one that produces random outputs of disease-positive or disease-negative outcomes) has an AUROC of 0.50.

The deep learning algorithm was evaluated at two classification thresholds of 0.5 and 0.9. At a threshold of 0.5, the deep learning algorithm achieved a sensitivity of 0.84, a specificity of 0.62, and a positive predictive value of 0.4. At a classification threshold of 0.9, the deep learning algorithm achieved a sensitivity of 0.6, a specificity of 0.92, and a positive predictive value of 0.70. These results and the numbers for true positives, true negatives, false positives, and false negatives are shown in Table 7. As expected, the performance of the deep learning algorithm performance is dependent on the selection of the classification threshold above which positive classifications are made.

TABLE 7
Algorithm performance metrics evaluated by setting the classification
threshold of the classifier to 0.5 and 0.9

| Threshold | 0.5 | 0.9 |
|---|---|---|
| True positives | 664 | 478 |
| True negatives | 1638 | 2430 |
| False positives | 997 | 205 |
| False negatives | 127 | 313 |
| Precision/PPV | 0.40 | 0.70 |
| Sensitivity | 0.84 | 0.60 |
| Specificity | 0.62 | 0.92 |

Discussion

This study demonstrated that a machine-learning classifier can be configured to generate predictions of sepsis onset based on a small set of inputs (e.g., 6 types of vital sign measurements) with high performance (e.g., an AUROC of 0.84). Open source datasets comprising stays by patients who were admitted to the ICU were used due to the availability of the data, and established that a deep learning algorithm can be configured to process an input set of 6 or fewer vital sign measurements to generate classifications of sepsis prediction with high performance. In particular, the trained deep learning model was capable of making predictions 8 hours before the onset of sepsis (with an AUROC of 0.73) that were more accurate than risk score-based sepsis assessments (e.g., as indicated by SOFA, qSOFA, SIRS, and MEWS scores) at the time that sepsis was already occurring. Therefore, deep-learning algorithms can be applied to predict sepsis using only a small subset of non-invasive physiological measurements (e.g., a plurality comprising no more than 6, no more than 5, no more than 4, no more than 3, no more than 2, or no more than 1 different types of non-invasive physiological measurements).

A comparison of recent approaches is presented in Table 8. These approaches include those described by, for example, (1) Moor et al., “Early Recognition of Sepsis with Gaussian Process Temporal Convolutional Networks and Dynamic Time Warping,” arxiv.org/abs/1902.01659, 2019; (2) Kaji et al., “An attention based deep learning model of clinical events in the intensive care unit,” PLoS One, 2019; (3) Futoma et al., “An Improved Multi-Output Gaussian Process RNN with Real-Time Validation for Early Sepsis Detection,” arxiv.org/abs/1708.05894, 2017; (4) Nemati et al., “An Interpretable Machine Learning Model for Accurate Prediction of Sepsis in the ICU,” Crit Care Med, 46(4):1, 2017; (5) Taylor et al., “Prediction of In-hospital Mortality in Emergency Department Patients With Sepsis: A Local Big Data-Driven, Machine Learning Approach,” Acad Emerg Med, 23(3):269-278, 2016; and (6) Desautels et al., “Prediction of Sepsis in the Intensive Care Unit With Minimal Electronic Health Record Data: A Machine Learning Approach,” JMIR Medical Informatics, 4(3):e28, 2016; each of which is incorporated herein by reference in its entirety.

TABLE 8
A comparison of selected machine learning approaches for sepsis prediction, including
those using minimal vital sign inputs or deep learning models to predict sepsis-positive
or sepsis-negative cases. The first column indicates the selected machine learning
approach, the second and third columns show whether the machine learning approach
uses deep learning and/or a minimal number of vital sign inputs, and the fourth and
fifth columns show more descriptions about the model and the inputs.

| Machine | | Use of at least | | |
|---|---|---|---|---|
| learning | | a small set of | | |
| approach for | Use of | non-invasive | Machine | Set of machine |
| prediction of | Deep | inputs (6 | learning | learning |
| sepsis | Learning | vital signs) | model used | model inputs |
| Methods of the | Yes | Yes | LSTM-RNN | 6 vital signs and |
| present | | | | demographics |
| disclosure | | | | |
| Moor et al. | Yes | No | MGP-TCN, MGP- | 44 vital and laboratory |
| 2019 | | | RNN, Raw-TCN, | parameters |
| | | | Dynamic Time | |
| | | | Warping (DTW)- | |
| | | | KNN (deep learning) | |
| Kaji et al. 2019 | Yes | No | LSTM with | 119 features including |
| | | | Attentional | complete blood count, |
| | | | Mechanism | vital signs, lab results, |
| | | | | demographic data, and |
| | | | | prescribed medications |
| Futoma et al. | Yes | No | RNN | 6 vitals, 28 laboratory |
| 2017 | | | | values, 35 covariate |
| | | | | inputs and administration |
| | | | | of antibiotics |
| Nemati et al. | No | No | Modified regularized | 65 inputs comprising |
| 2017 | | | Weilbull-Cox | vital-signs, lab tests, |
| | | | | demographics and |
| | | | | historical features |
| Taylor et al. | No | No | Random Forest | 566 variables consisting |
| 2016 | | | | of ED procedures, |
| | | | | laboratory results, |
| | | | | vital signs, |
| | | | | demographics, |
| | | | | medical history, |
| | | | | nursing information, |
| | | | | and medications |
| Desautels et al. | No | Yes | InSight (Bayesian | 6 vital signs and Glasgow |
| 2016 | | | Inference) | Coma Score (GCS) |

Early detection is particularly important in clinical treatment of sepsis cases, since the rate of mortality for a given sepsis patient increases with each hour until antibiotics are administered to the sepsis patient. Though this retrospective analysis was performed on patient stays from the ICU, the deep learning algorithm was configured to process a small input set of vital sign measurements that were collected non-invasively and routinely measured in a hospital or other clinical setting. Using appropriate signal processing and characterization, this model may be translated to the hospital or other clinical setting where these physiological measurements are recorded. The small subset of inputs used here may be used to train a deep learning algorithm to achieve high-performance classifications of predictions (e.g., as measured by metrics such as sensitivity, specificity, positive predictive value, negative predictive value, AUROC, AUPRC, or a combination thereof) of sepsis without the need to incorporate clinical variables such as antibiotic administration, bedside scores, laboratory measurements, and patient clinical history. Despite this, the performance of the deep learning algorithm was comparable with deep learning models having more expansive sets of input features, in the metrics of AUROC and AUPRC. One or more additional types of clinical data (e.g., antibiotic administration, bedside scores, laboratory measurements, and patient clinical history) may be added to the set of input data for the deep learning algorithm to further optimize the performance metrics of the sepsis prediction (e.g., sensitivity, specificity, positive predictive value, negative predictive value, AUROC, AUPRC, or a combination thereof), as desired.

The performance of the deep learning algorithm was presented in part using the measure of AUPRC as a supplement to the AUROC. While AUROC is commonly used as a standard performance measure of diagnostic tests, it may fail to address an issue of the class imbalance between the set of sepsis patients and the set of non-sepsis patients. The vast majority of sepsis patient cases used to train and validate the deep learning algorithm do not meet sepsis criteria during their hospital stay. As a result, there are much fewer patients who are positive for the disease than those who are negative for the disease. The AUROC is based on the ability of the diagnostic test to identify true negatives; in contrast, the AUPRC characterizes the ability of the diagnostic test to identify true positives. In the case of a class imbalance, as was present in this study of sepsis cases, very high AUROC scores may be reported for a given classifier despite having a poor precision or positive predictive value, due to an overabundance of disease-negative cases and very few disease-positive cases.

The deep learning algorithm, as demonstrated by an example retrospective study, may be improved by performing an observational prospective validation which uses the Sepsis-3 criteria to label the full spectrum of patients contained within the database. We suggest that an observational prospective validation is a next step to be conducted to demonstrate the capability of the algorithm.

Further, labeling of the onset of sepsis may be adjusted as needed, given that a clinical consensus may not be available, yet is critical to the development of machine learning models. For example, any errors of the selected criteria for determining sepsis onset can magnify errors in the deep learning model on which it is trained. The Sepsis-3 definition selected for this study may not be the clinical definition used in all medical centers; some of which may use terms such as sepsis, severe sepsis and septic shock as part of the Sepsis-2 criteria. Similarly, ICD codes may be used to label sepsis occurrence, but may encounter challenges from a lack of precise temporal information and problems inherent with bias in claims and billing codes. Comparisons of relative performance of different machine learning approaches may be improved or refined by using a set of standardized criteria for labeling sepsis-positive patient cases.

In some embodiments, deep learning algorithms may be further improved or refined by being trained on training data sets comprising different patient groups to reduce, minimize, or eliminate confounding factors that may exist between patients with varying conditions. Further, as the open-source databases selected comprise ICU data, the time horizons before sepsis that were examined were restricted to ten hours beforehand. With general ward data, models may be configured to generate predictions at time horizons earlier than 8 hours before sepsis onset, such as by incorporating training data sets comprising such earlier time horizons.

The deep learning model may be tuned such that the sensitivity, specificity, positive predictive value, negative predictive value, AUROC, AUPRC, or a combination thereof, can be adjusted. For example, the classification threshold of the deep learning model may be adjusted based on the expected clinical use or application. For example, the classification threshold may be set at a high value (e.g., about 0.70, about 0.75, about 0.80, 0.85, about 0.90, about 0.95, or about 0.99), such that only the most at-risk patients for sepsis are assigned a sepsis-positive outcome and a corresponding alert. This high-specificity model may be incorporated, for example, into antibiotics stewardship programs that are seeking to determine the optimal dosage and use of antibiotics. As another example, the classification threshold may be set at a lower value (e.g., about 0.25, about 0.30, about 0.35, about 0.40, about 0.45, about 0.50, about 0.55, about 0.60, or about 0.65) for certain cases where a high-sensitivity model is desired.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-104. (canceled)

105. A system for monitoring a subject, comprising:
one or more sensors comprising an electrocardiogram (ECG) sensor, which one or more sensors are configured to acquire health data comprising a plurality of vital sign measurements of the subject over a period of time; and
a mobile electronic device, comprising:
an electronic display;
a wireless transceiver; and
one or more computer processors operatively coupled to the electronic display and the wireless transceiver, which one or more computer processors are configured to (i) receive the health data from the one or more sensors through the wireless transceiver, (ii) process the health data using a trained algorithm to generate an output indicative of a progression or regression of sepsis of the subject over the period of time at an Area Under the Receiver Operating Characteristic (AUROC) of at least about 0.70, and (iii) provide the output for display to the subject on the electronic display.

106. The system of claim 105, wherein the ECG sensor comprises one or more ECG electrodes.

107. The system of claim 105, wherein the plurality of vital sign measurements comprises one or more measurements selected from the group consisting of heart rate, heart rate variability, systolic blood pressure, diastolic blood pressure, respiratory rate, blood oxygen concentration (SpO₂), carbon dioxide concentration in respiratory gases, a hormone level, sweat analysis, blood glucose, body temperature, impedance, conductivity, capacitance, resistivity, electromyography, galvanic skin response, neurological signals, and immunology markers.

108. The system of claim 105, wherein the plurality of vital sign measurements comprises no more than 10 types of vital sign measurements selected from the group consisting of heart rate, heart rate variability, systolic blood pressure, diastolic blood pressure, respiratory rate, blood oxygen concentration (SpO₂), carbon dioxide concentration in respiratory gases, a hormone level, sweat analysis, blood glucose, body temperature, impedance, conductivity, capacitance, resistivity, electromyography, galvanic skin response, neurological signals, and immunology markers.

109. The system of claim 108, wherein the plurality of vital sign measurements comprises no more than 6 types of vital sign measurements, and wherein the 6 types of vital sign measurements are heart rate, respiratory rate, body temperature, systolic blood pressure, diastolic blood pressure, and blood oxygen.

110. The system of claim 105, wherein the one or more computer processors are further configured to (i) present an alert on the electronic display based at least on the output, or (ii) transmit the alert over a network to a health care provider of the subject based at least on the output.

111. The system of claim 105, wherein the trained algorithm comprises a machine learning-based classifier configured to process the health data to generate the output indicative of the progression or regression of the sepsis of the subject.

112. The system of claim 105, wherein the machine learning-based classifier is selected from the group consisting of a support vector machine (SVM), a naïve Bayes classification, a random forest, a neural network, a deep neural network (DNN), a recurrent neural network (RNN), a deep RNN, a long short-term memory (LSTM) recurrent neural network (RNN), and a gated recurrent unit (GRU) recurrent neural network (RNN).

113. The system of claim 112, wherein the trained algorithm comprises a recurrent neural network (RNN).

114. The system of claim 112, wherein the trained algorithm comprises a long short-term memory (LSTM) recurrent neural network (RNN).

115. The system of claim 105, wherein (i) the subject is being monitored for post-surgery complications, or (ii) the subject has received a treatment comprising a bone marrow transplant or an active chemotherapy, and the subject is being monitored for post-treatment complications.

116. The system of claim 105, wherein the period of time includes a window beginning about 2 hours prior to the onset of the sepsis and ending at the onset of the sepsis.

117. The system of claim 105, wherein the period of time includes a window beginning about 4 hours prior to the onset of the sepsis and ending at about 2 hours prior to the onset of the sepsis.

118. The system of claim 105, wherein the period of time includes a window beginning about 6 hours prior to the onset of the sepsis and ending at about 4 hours prior to the onset of the sepsis.

119. The system of claim 105, wherein the period of time includes a window beginning about 8 hours prior to the onset of the sepsis and ending at about 6 hours prior to the onset of the sepsis.

120. The system of claim 105, wherein the period of time includes a window beginning about 10 hours prior to the onset of the sepsis and ending at about 8 hours prior to the onset of the sepsis.

121. The system of claim 105, wherein the one or more computer processors are configured to process the health data using the trained algorithm to generate the output indicative of the progression or regression of the sepsis of the subject over the period of time at an Area Under the Precision-Recall Curve (AUPRC) of at least 0.40.

122. The system of claim 105, wherein the one or more computer processors are configured to process the health data using the trained algorithm to generate the output indicative of the progression or regression of the sepsis of the subject over the period of time with a specificity of at least about 40%.

123. A method for monitoring a subject, comprising:
(a) receiving, using a wireless transceiver of a mobile electronic device of the subject, health data from one or more sensors, which one or more sensors comprise an electrocardiogram (ECG) sensor, which health data comprises a plurality of vital sign measurements of the subject over a period of time;
(b) using one or more programmed computer processors of the mobile electronic device to process the health data using a trained algorithm to generate an output indicative of a progression or regression of sepsis of the subject over the period of time at an area under the receiver operating characteristic (AUROC) of at least about 0.70; and
(c) presenting the output for display on an electronic display of the mobile electronic device.

124. A system for monitoring a subject, comprising:
a communications interface in network communication with a mobile electronic device of a user, wherein the communication interface receives from the mobile electronic device health data collected from a subject using one or more sensors, which one or more sensors comprise an electrocardiogram (ECG) sensor, wherein the health data comprises a plurality of vital sign measurements of the subject over a period of time;
one or more computer processors operatively coupled to the communications interface, wherein the one or more computer processors are individually or collectively programmed to (i) receive the health data from the communications interface, (ii) use a trained algorithm to analyze the health data to generate an output indicative of a progression or regression of sepsis of the subject over the period of time at an Area Under the Receiver Operating Characteristic (AUROC) of at least about 0.70, and (iii) direct the output to the mobile electronic device over the network.
